SEQUENCE LISTING (60 sequences)

SEQ ID NO: 1 - protein, 5 aa, Artificial Sequence
  heavy chain complementarity determining region 1
SYDMT

SEQ ID NO: 2 - protein, 16 aa, Artificial Sequence
  heavy chain complementarity determining region 2
IIYTGGLTYYASWAKG

SEQ ID NO: 3 - protein, 8 aa, Artificial Sequence
  heavy chain complementarity determining region 3
EVGDNNNF

SEQ ID NO: 4 - protein, 11 aa, Artificial Sequence
  light chain complementarity determining region 1
QASESVDSGLA

SEQ ID NO: 5 - protein, 7 aa, Artificial Sequence
  light chain complementarity determining region 2
DDTDLAS

SEQ ID NO: 6 - protein, 12 aa, Artificial Sequence
  light chain complementarity determining region 3
QYTDYGRTYVGT

SEQ ID NO: 7 - protein, 113 aa, Artificial Sequence
  heavy chain variable region
QSLEESGGRLVKPDETLTITCTVSGIDLSSYDMTWVRQAPGKGLEWIGIIYTGGLTYYASWAKGRFTISKTSTTVDLKITSPTTEDTATYFCAREVGDNNNFWGPGTLVTVSS

SEQ ID NO: 8 - protein, 110 aa, Artificial Sequence
  light chain variable region
DVVMTQTPASVSEPVGGTVTIKCQASESVDSGLAWYHQKPGQPPKLLIYDDTDLASGVPSRFKGSRSGTAHTLTISDLECADAATYYCQYTDYGRTYVGTFGGGTEVVIK

SEQ ID NO: 9 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 10 - DNA, 1431 bp, Artificial Sequence
  Siglec-9 DNA sequence used for genetic immunization
cctgcaggccgctagcaccatgctgctgctgctgctgcccctgctgtggggccgcgagcgcgccgagggccagacctccaagctgctgaccatgcagtcctccgtgaccgtgcaggagggcctgtgcgtgcacgtgccctgctccttctcctacccctcccacggctggatctaccccggccccgtggtgcacggctactggttccgcgagggcgccaacaccgaccaggacgcccccgtggccaccaacaaccccgcccgcgccgtgtgggaggagacccgcgaccgcttccacctgctgggcgacccccacaccaagaactgcaccctgtccatccgcgacgcccgccgctccgacgccggccgctacttcttccgcatggagaagggctccatcaagtggaactacaagcaccaccgcctgtccgtgaacgtgaccgccctgacccaccgccccaacatcctgatccccggcaccctggagtccggctgcccccagaacctgacctgctccgtgccctgggcctgcgagcagggcaccccccccatgatctcctggatcggcacctccgtgtcccccctggacccctccaccacccgctcctccgtgctgaccctgatcccccagccccaggaccacggcacctccctgacctgccaggtgaccttccccggcgcctccgtgaccaccaacaagaccgtgcacctgaacgtgtcctaccccccccagaacctgaccatgaccgtgttccagggcgacggcaccgtgtccaccgtgctgggcaacggctcctccctgtccctgcccgagggccagtccctgcgcctggtgtgcgccgtggacgccgtggactccaacccccccgcccgcctgtccctgtcctggcgcggcctgaccctgtgcccctcccagccctccaaccccggcgtgctggagctgccctgggtgcacctgcgcgacgccgccgagttcacctgccgcgcccagaaccccctgggctcccagcaggtgtacctgaacgtgtccctgcagtccaaggccacctccggcgtgacccagggcgtggtgggcggcgccggcgccaccgccctggtgttcctgtccttctgcgtgatcttcgtggtggtgcgctcctgccgcaagaagtccgcccgccccgccgccggcgtgggcgacaccggcatcgaggacgccaacgccgtgcgcggctccgcctcccagggccccctgaccgagccctgggccgaggactccccccccgaccagcccccccccgcctccgcccgctcctccgtgggcgagggcgagctgcagtacgcctccctgtccttccagatggtgaagccctgggactcccgcggccaggaggccaccgacaccgagtactccgagatcaagatccaccgctaatctagaacgcgtttaattaa

SEQ ID NO: 11 - protein, 556 aa, Artificial Sequence
  Siglec-9-Fc protein used for the protein immunization
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLS

SEQ ID NO: 12 - protein, 5 aa, Artificial Sequence
  heavy chain complementarity determining region 1
SYWMH

SEQ ID NO: 13 - protein, 17 aa, Artificial Sequence
  heavy chain complementarity determining region 2
EINPSNGHTNYNEKFES

SEQ ID NO: 14 - protein, 13 aa, Artificial Sequence
  heavy chain complementarity determining region 3
GVESYDFDDALDY

SEQ ID NO: 15 - protein, 11 aa, Artificial Sequence
  light chain complementarity determining region 1
RASQDINNYLN

SEQ ID NO: 16 - protein, 7 aa, Artificial Sequence
  light chain complementarity determining region 2
YTSRLHS

SEQ ID NO: 17 - protein, 9 aa, Artificial Sequence
  light chain complementarity determining region 3
QQGNTLPFT

SEQ ID NO: 18 - protein, 122 aa, Artificial Sequence
  heavy chain variable region
QVQLQQPGAELVKPGSPVKLSCKASYFTFTSYWMHWVRQRPGQGLEWIGEINPSNGHTNYNEKFESKATLTVDRSSSTAYMQLSSLTSEDSAVFYCANGVESYDFDDALDYWGQGTSVTVSS

SEQ ID NO: 19 - protein, 107 aa, Artificial Sequence
  light chain variable region
DIQMTQTTSSLSASLGDRVTISCRASQDINNYLNWYQQKPDGTIKLLIYYTSRLHSGVPSRFSGSGSGTDYSLTINNLEQEDIATYFCQQGNTLPFTFGGGTKLEIK

SEQ ID NO: 20 - protein, 6 aa, Artificial Sequence
  heavy chain complementarity determining region 1
TDYNIH

SEQ ID NO: 21 - protein, 17 aa, Artificial Sequence
  heavy chain complementarity determining region 2
YIYPYNGDTGYNQKFQN

SEQ ID NO: 22 - protein, 11 aa, Artificial Sequence
  heavy chain complementarity determining region 3
GNYLYYYAMDY

SEQ ID NO: 23 - protein, 16 aa, Artificial Sequence
  light chain complementarity determining region 1
SSSQSLVHSNGNTYLH

SEQ ID NO: 24 - protein, 7 aa, Artificial Sequence
  light chain complementarity determining region 2
KVSNRFS

SEQ ID NO: 25 - protein, 9 aa, Artificial Sequence
  light chain complementarity determining region 3
SQNTHVPLT

SEQ ID NO: 26 - protein, 120 aa, Artificial Sequence
  heavy chain variable region
EVQLQQSGPELVKPGASVRISCKASGYTFTDYNIHWVKQSQGKSLEWIGYIYPYNGDTGYNQKFQNKATLTVDNSSSTAYMELRSLTSEDSAVYYCANGNYLYYYAMDYWGQGTSVTVSS

SEQ ID NO: 27 - protein, 112 aa, Artificial Sequence
  light chain variable region
DVVMTQTPLSLPVSLGDQVSISCSSSQSLVHSNGNTYLHWYLQKSGQSPKLLIYKVSNRFSGVPDRFRGSGSGTDFTLKISRVEAEDLGVYFCSQNTHVPLTFGAGTKLELK

SEQ ID NO: 28 - protein, 436 aa, Artificial Sequence
  full length heavy chain
QSLEESGGRLVKPDETLTITCTVSGIDLSSYDMTWVRQAPGKGLEWIGIIYTGGLTYYASWAKGRFTISKTSTTVDLKITSPTTEDTATYFCAREVGDNNNFWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 29 - protein, 214 aa, Artificial Sequence
  full length light chain
DVVMTQTPASVSEPVGGTVTIKCQASESVDSGLAWYHQKPGQPPKLLIYDDTDLASGVPSRFKGSRSGTAHTLTISDLECADAATYYCQYTDYGRTYVGTFGGGTEVVIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 30 - protein, 5 aa, Artificial Sequence
  heavy chain complementarity determining region 1
SYYMS

SEQ ID NO: 31 - protein, 16 aa, Artificial Sequence
  heavy chain complementarity determining region 2
YIWTDGSAHYATWTKG

SEQ ID NO: 32 - protein, 14 aa, Artificial Sequence
  heavy chain complementarity determining region 3
ANSGSHTYIEKFNL

SEQ ID NO: 33 - protein, 11 aa, Artificial Sequence
  light chain complementarity determining region 1
QASPSINDELS

SEQ ID NO: 34 - protein, 7 aa, Artificial Sequence
  light chain complementarity determining region 2
EASALES

SEQ ID NO: 35 - protein, 15 aa, Artificial Sequence
  light chain complementarity determining region 3
QCTYGSRSDISYGNT

SEQ ID NO: 36 - protein, 120 aa, Artificial Sequence
  heavy chain variable region
QSVEESGGRLVTPETPLTLTCTASGFSLSSYYMSWVRQAPGKGLEWIGYIWTDGSAHYATWTKGRFTISKTSSTTVDLKITSPTTEDTATYFCVRANSGSHTYIEKFNLWGPGTLVTVSS

SEQ ID NO: 37 - protein, 114 aa, Artificial Sequence
  light chain variable region
DVVMTQTPASVEATVGGSVTIKCQASPSINDELSWYQQKPGQRPTLLIYEASALESGVPSRFKGSGSGTEFTLTIGDLECADAATYYCQCTYGSRSDISYGNTFGGGTEVVVKG

SEQ ID NO: 38 - protein, 443 aa, Artificial Sequence
  full length heavy chain
QSVEESGGRLVTPETPLTLTCTASGFSLSSYYMSWVRQAPGKGLEWIGYIWTDGSAHYATWTKGRFTISKTSSTTVDLKITSPTTEDTATYFCVRANSGSHTYIEKFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 39 - protein, 217 aa, Artificial Sequence
  Full length light chain
DVVMTQTPASVEATVGGSVTIKCQASPSINDELSWYQQKPGQRPTLLIYEASALESGVPSRFKGSGSGTEFTLTIGDLECADAATYYCQCTYGSRSDISYGNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 40 - protein, 452 aa, Artificial Sequence
  full length heavy chain
QVQLQQPGAELVKPGSPVKLSCKASYFTFTSYWMHWVRQRPGQGLEWIGEINPSNGHTNYNEKFESKATLTVDRSSSTAYMQLSSLTSEDSAVFYCANGVESYDFDDALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, Artificial Sequence
  full length light chain
DIQMTQTTSSLSASLGDRVTISCRASQDINNYLNWYQQKPDGTIKLLIYYTSRLHSGVPSRFSGSGSGTDYSLTINNLEQEDIATYFCQQGNTLPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 443 aa, Artificial Sequence
  full length heavy chain
EVQLQQSGPELVKPGASVRISCKASGYTFTDYNIHWVKQSQGKSLEWIGYIYPYNGDTGYNQKFQNKATLTVDNSSSTAYMELRSLTSEDSAVYYCANGNYLYYYAMDYWGQGTSVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 43 - protein, 216 aa, Artificial Sequence
  full length light chain
DVVMTQTPLSLPVSLGDQVSISCSSSQSLVHSNGNTYLHWYLQKSGQSPKLLIYKVSNRFSGVPDRFRGSGSGTDFTLKISRVEAEDLGVYFCSQNTHVPLTFGAGTKLELKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 44 - protein, 339 aa, Artificial Sequence
  Siglec-9 fused to a C-terminal his-tag
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVDHHHHHH

SEQ ID NO: 45 - protein, 16 aa, Artificial Sequence
  heavy chain complementarity determining region 2
IIYTGGLTYYADSVKG

SEQ ID NO: 46 - protein, 11 aa, Artificial Sequence
  light chain complementarity determining region 1
RASESVSSGLA

SEQ ID NO: 47 - protein, 116 aa, Artificial Sequence
  heavy chain variable region
EVQLVESGGGLVQPGGSLRISCAASGIDLSSYDMTWVRQAPGKGLEWVSIIYTGGLTYYADSVKGRFTISRDNSKNTVYLQMNSLRAEDTAVYYCAREVGDNNNFWGQGTLVTVSS

SEQ ID NO: 48 - protein, 110 aa, Artificial Sequence
  light chain variable region
DIVMTQSPSSVSASVGDRVTITCRASESVSSGLAWYQQKPGKAPKLLVYDDTDLASGVPSRFSGSGSGTEFTLTISSLQPDDFATFYCQYTDYGRTYVGTFGGGTKVEIK

SEQ ID NO: 49 - protein, 446 aa, Artificial Sequence
  full length heavy chain
EVQLVESGGGLVQPGGSLRISCAASGIDLSSYDMTWVRQAPGKGLEWVSIIYTGGLTYYADSVKGRFTISRDNSKNTVYLQMNSLRAEDTAVYYCAREVGDNNNFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 217 aa, Artificial Sequence
  full length light chain
DIVMTQSPSSVSASVGDRVTITCRASESVSSGLAWYQQKPGKAPKLLVYDDTDLASGVPSRFSGSGSGTEFTLTISSLQPDDFATFYCQYTDYGRTYVGTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 16 aa, Artificial Sequence
  heavy chain complementarity determining region 2
  VARIANT (12)..(12): /replace=“Ser”
  VARIANT (13)..(13): /replace=“Trp”
  VARIANT (14)..(14): /replace=“Ala”
IIYTGGLTYYADSVKG

SEQ ID NO: 52 - protein, 11 aa, Artificial Sequence
  light chain complementarity determining region 1
  VARIANT (1)..(1): /replace=“Gln”
  VARIANT (7)..(7): /replace=“Asp”
RASESVSSGLA

SEQ ID NO: 53 - protein, 124 aa, Artificial Sequence
  heavy chain variable region
EVQLVESGGGLVQPGGSLRISCAASGFTFSSYAMSWVRQAPGKGLEWVSSISDSGVRTYYSDSVKGRFTISRDNSKNTVYLQMNSLRAEDTAVYYCAKFVGSYYDYWSGSFDYWGQGTLVTVSS

SEQ ID NO: 54 - protein, 107 aa, Artificial Sequence
  light chain variable region
DIVMTQSPSSVSASVGDRVTITCRASQTISSWLAWYQQKPGKAPKLLVYKASTLESGVPSRFSGSGSGTEFTLTISSLQPDDFATFYCQQYNTYPLTFGGGTKVEIK

SEQ ID NO: 55 - protein, 94 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSY

SEQ ID NO: 56 - protein, 12 aa, Homo sapiens
LTFGGGTKVEIK

SEQ ID NO: 57 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 58 - protein, 13 aa, Homo sapiens
DYWGQGTLVTVSS

SEQ ID NO: 59 - protein, 560 aa, Artificial Sequence
  Siglec-7-Fc
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLS

SEQ ID NO: 60 - protein, 467 aa, Homo sapiens
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

(19) United States
(12) Patent Application Publication
(10) Pub. No.: US 2023/0009582 A1
(43) Pub. Date: Jan. 12, 2023

(54) ANTI-SIGLEC-9 ANTIBODY MOLECULES

(71) Applicants: MEMO THERAPEUTICS AG, Schlieren (CH); UNIVERSITÄT BASEL, Basel (CH)

(72) Inventors: Heinz LÄUBLI, Binningen (CH); Simone SCHMITT, Dietikon (CH); Christoph ESSLINGER, Zürich (CH)

(21) Appl. No.: 17/776,980

(22) PCT Filed: Nov. 13, 2020
(86) PCT No.: PCT/EP2020/082072

§ 371 Date: May 13, 2022

(30) Foreign Application Priority Data
Nov. 14, 2019 (EP) 19209100.7
Nov. 14, 2019 (EP) 19209104.9

(51) Int. Cl. C07K 16/28; C12N 15/63; A61P 35/00

(52) U.S. Cl. CPC C07K 16/2851; C12N 15/63; A61P 35/00; C07K 2317/565; C07K 2317/24; C07K 2317/92

(57) ABSTRACT

Anti-Siglec-9 antibody molecules or binding fragments thereof are disclosed. These Anti-Siglec-9 antibody molecules or binding fragments can be used to treat cancer, acute or chronic hepatitis B.

### FIELD

The present disclosure relates to anti-Siglec-9 antibody molecules or binding fragments thereof. The present disclosure further relates to nucleic acids encoding the antibody molecules or binding fragments thereof, expression vectors, host cells and methods for making the antibody molecules or binding fragments thereof. Pharmaceutical compositions comprising the antibody molecules or binding fragments thereof are also provided. The anti-Siglec-9 antibody molecules or binding fragments thereof of the present disclosure can be used (alone or in combination with other agents or therapeutic modalities) to treat cancer, acute or chronic hepatitis B. Thus, the present disclosure further relates to anti-Siglec-9 antibody molecules or binding fragments thereof, or pharmaceutical compositions comprising anti-Siglec-9 antibody molecules or binding fragments thereof, for use in the treatment of cancer, acute or chronic hepatitis B.

### BACKGROUND

During malignant transformation, glycosylation is heavily altered compared with healthy tissue due to differential expression of e.g. glycosyltransferases, glycosidases and monosaccharide transporters within the cancer microenvironment. The upregulation and alteration of terminal sialic acid structures is a hallmark of cancer.

Sialic acid structures patterns are recognized through recognition receptors called Siglecs. Siglecs are sialic acid structure-binding immuno-modulatory, often inhibitory receptors expressed preferentially on immune cells. In recent years, several experimental models have provided evidence that Siglecs are implicated in cancer progression and immune evasion. These experimental models also suggest that Siglecs, such as e.g. Siglec-9, could be targeted to improve anti-tumor immunity.

In view of the ongoing need for improved strategies for targeting diseases such as cancer, new compositions for regulating Siglec-9 activity are highly desirable.

### SUMMARY

### Aspect A

In one aspect A1, the present disclosure relates to an anti-Siglec-9 antibody molecule or an anti-Siglec-9 binding fragment thereof.

### Structural Properties

In some embodiments of aspect A1, the present disclosure relates to an anti-Siglec-9 antibody molecule or an anti-Siglec-9 binding fragment thereof, wherein the antibody molecule or binding fragment comprises at least one, two, three, four, five or six complementarity determining regions (CDRs) (or collectively all of the CDRs) from a heavy chain variable region (VH) and/or a light chain variable region (VL) comprising an amino acid sequence shown in Table 6, wherein one or more of the CDRs (or collectively all of the CDRs) may have one, two, three, four, five, six or more changes, e.g., amino acid substitutions (e.g., conservative amino acid substitutions), insertions or deletions, relative to an amino acid sequence shown in Table 6.

In some embodiments of aspect A1, the present disclosure relates to an anti-Siglec-9 antibody molecule or an anti-Siglec-9 binding fragment thereof, wherein the antibody molecule or binding fragment comprises at least one, two, three, four, five or six complementarity determining regions (CDRs) (or collectively all of the CDRs) from a heavy chain variable region (VH) and/or a light chain variable region (VL) comprising an amino acid sequence shown in Table 5, wherein one or more of the CDRs (or collectively all of the CDRs) may have one, two, three, four, five, six or more changes, e.g., amino acid substitutions (e.g., conservative amino acid substitutions), insertions or deletions, relative to an amino acid sequence shown in Table 5.

In some embodiments of aspect A1, the present disclosure relates to an anti-Siglec-9 antibody molecule or an anti-Siglec-9 binding fragment thereof, wherein the antibody molecule or binding fragment comprises at least one, two, three, four, five or six complementarity determining regions (CDRs) (or collectively all of the CDRs) from a heavy chain variable region (VH) and/or a light chain variable region (VL) comprising an amino acid sequence shown in Table 1, wherein one or more of the CDRs (or collectively all of the CDRs) may have one, two, three, four, five, six or more changes, e.g., amino acid substitutions (e.g., conservative amino acid substitutions), insertions or deletions, relative to an amino acid sequence shown in Table 1.

In some embodiments of aspect A1, the present disclosure relates to an anti-Siglec-9 antibody molecule or an anti-Siglec-9 binding fragment thereof, comprising:

a heavy chain variable region (VH) comprising one, two, or three of: a heavy chain complementarity determining region 1 (VHCDR1) amino acid sequence of SEQ ID NO: 1 or a sequence having one, two, three, or four amino acid substitutions (e.g., conservative amino acid substitutions), insertions or deletions, a heavy chain complementarity determining region 2 (VHCDR2) amino acid sequence of SEQ ID NO: 51 or a sequence having one, two, three, or four amino acid substitutions (e.g., conservative amino acid substitutions), insertions or deletions, and a heavy chain complementarity determining region 3 (VHCDR3) amino acid sequence of SEQ ID NO: 3 or a sequence having one, two, three, or four amino acid substitutions (e.g., conservative amino acid substitutions), insertions or deletions; and/or a light chain variable region (VL) comprising one, two, or three of: a light chain complementarity determining region 1 (VLCDR1) amino acid sequence of SEQ ID NO: 52 or a sequence having one, two, three, or four amino acid substitutions (e.g., conservative amino acid substitutions), insertions or deletions, a light chain complementarity determining region 2 (VLCDR2) amino acid sequence of SEQ ID NO: 5 or a sequence having one, two, three, or four amino acid substitutions (e.g., conservative amino acid substitutions), insertions or deletions, and a light chain complementarity determining region 3 (VLCDR3) amino acid sequence of SEQ ID NO: 6 or a sequence having one, two, three, or four amino acid substitutions (e.g., conservative amino acid substitutions), insertions or deletions.

In some embodiments of aspect A1, the present disclosure relates to an anti-Siglec-9 antibody molecule or an anti-Siglec-9 binding fragment thereof, comprising:

a heavy chain variable region (VH) comprising one, two, or three of: a heavy chain complementarity determining region 1 (VHCDR1) amino acid sequence of SEQ ID NO: 1 or a sequence having one, two, three, or four amino acid substitutions (e.g., conservative amino acid substitutions), insertions or deletions, a heavy chain complementarity determining region 2 (VHCDR2) amino acid sequence of SEQ ID NO: 45 or a sequence having one, two, three, or four amino acid substitutions (e.g., conservative amino acid substitutions), insertions or deletions, and a heavy chain complementarity determining region 3 (VHCDR3) amino acid sequence of SEQ ID NO: 3 or a sequence having one, two, three, or four amino acid substitutions (e.g., conservative amino acid substitutions), insertions or deletions; and/or a light chain variable region (VL) comprising one, two, or three of: a light chain complementarity determining region 1 (VLCDR1) amino acid sequence of SEQ ID NO: 46 or a sequence having one, two, three, or four amino acid substitutions (e.g., conservative amino acid substitutions), insertions or deletions, a light chain complementarity determining region 2 (VLCDR2) amino acid sequence of SEQ ID NO: 5 or a sequence having one, two, three, or four amino acid substitutions (e.g., conservative amino acid substitutions), insertions or deletions, and a light chain complementarity determining region 3 (VLCDR3) amino acid sequence of SEQ ID NO: 6 or a sequence having one, two, three, or four amino acid substitutions (e.g., conservative amino acid substitutions), insertions or deletions.

In some embodiments of aspect A1, the present disclosure relates to an anti-Siglec-9 antibody molecule or an anti-Siglec-9 binding fragment thereof, comprising:

a heavy chain variable region (VH) comprising one, two, or three of: a heavy chain complementarity determining region 1 (VHCDR1) amino acid sequence of SEQ ID NO: 1 or a sequence having one, two, three, or four amino acid substitutions (e.g., conservative amino acid substitutions), insertions or deletions, a heavy chain complementarity determining region 2 (VHCDR2) amino acid sequence of SEQ ID NO: 2 or a sequence having one, two, three, or four amino acid substitutions (e.g., conservative amino acid substitutions), insertions or deletions, and a heavy chain complementarity determining region 3 (VHCDR3) amino acid sequence of SEQ ID NO: 3 or a sequence having one, two, three, or four amino acid substitutions (e.g., conservative amino acid substitutions), insertions or deletions; and/or a light chain variable region (VL) comprising one, two, or three of: a light chain complementarity determining region 1 (VLCDR1) amino acid sequence of SEQ ID NO: 4 or a sequence having one, two, three, or four amino acid substitutions (e.g., conservative amino acid substitutions), insertions or deletions, a light chain complementarity determining region 2 (VLCDR2) amino acid sequence of SEQ ID NO: 5 or a sequence having one, two, three, or four amino acid substitutions (e.g., conservative amino acid substitutions), insertions or deletions, and a light chain complementarity determining region 3 (VLCDR3) amino acid sequence of SEQ ID NO: 6 or a sequence having one, two, three, or four amino acid substitutions (e.g., conservative amino acid substitutions), insertions or deletions.

In some embodiments of aspect A1, the present disclosure relates to an anti-Siglec-9 antibody molecule or an anti-Siglec-9 binding fragment thereof comprising:

a heavy chain variable region (VH) comprising a heavy chain complementarity determining region 3 (VHCDR3) amino acid sequence of SEQ ID NO: 3, or a sequence having one, two, three, or four amino acid substitutions (e.g., conservative amino acid substitutions); and/or

a light chain variable region (VL) comprising a light chain complementarity determining region 3 (VLCDR3) amino acid sequence of SEQ ID NO: 6, or a sequence having one, two, three, or four amino acid substitutions (e.g., conservative amino acid substitutions).

In some embodiments of aspect A1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 1 or a sequence having one, two, three, or four amino acid substitutions (e.g., conservative amino acid substitutions), a heavy chain complementarity determining region 2 (VHCDR2) amino acid sequence of SEQ ID NO: 51 or a sequence having one, two, three, or four amino acid substitutions (e.g., conservative amino acid substitutions), and a heavy chain complementarity determining region 3 (VHCDR3) amino acid sequence of SEQ ID NO: 3 or a sequence having one, two, three, or four amino acid substitutions (e.g., conservative amino acid substitutions); and/or

a light chain variable region (VL) comprising a light chain complementarity determining region 1 (VLCDR1) amino acid sequence of SEQ ID NO: 52 or a sequence having one, two, three, or four amino acid substitutions (e.g., conservative amino acid substitutions), a light chain complementarity determining region 2 (VLCDR2) amino acid sequence of SEQ ID NO: 5 or a sequence having one, two, three, or four amino acid substitutions (e.g., conservative amino acid substitutions), and a light chain complementarity determining region 3 (VLCDR3) amino acid sequence of SEQ ID NO: 6 or a sequence having one, two, three, or four amino acid substitutions (e.g., conservative amino acid substitutions).

In some embodiments of aspect A1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 1 or a sequence having one, two, three, or four amino acid substitutions (e.g., conservative amino acid substitutions), a heavy chain complementarity determining region 2 (VHCDR2) amino acid sequence of SEQ ID NO: 45 or a sequence having one, two, three, or four amino acid substitutions (e.g., conservative amino acid substitutions), and a heavy chain complementarity determining region 3 (VHCDR3) amino acid sequence of SEQ ID NO: 3 or a sequence having one, two, three, or four amino acid substitutions (e.g., conservative amino acid substitutions); and/or

a light chain variable region (VL) comprising a light chain complementarity determining region 1 (VLCDR1) amino acid sequence of SEQ ID NO: 46 or a sequence having one, two, three, or four amino acid substitutions (e.g., conservative amino acid substitutions), a light chain complementarity determining region 2 (VLCDR2) amino acid sequence of SEQ ID NO: 5 or a sequence having one, two, three, or four amino acid substitutions (e.g., conservative amino acid substitutions), and a light chain complementarity determining region 3 (VLCDR3) amino acid sequence of SEQ ID NO: 6 or a sequence having one, two, three, or four amino acid substitutions (e.g., conservative amino acid substitutions).

In some embodiments of aspect A1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 1 or a sequence having one, two, three, or four amino acid substitutions (e.g., conservative amino acid substitutions), a heavy chain complementarity determining region 2 (VHCDR2) amino acid sequence of SEQ ID NO: 2 or a sequence having one, two, three, or four amino acid substitutions (e.g., conservative amino acid substitutions), and a heavy chain complementarity determining region 3 (VHCDR3) amino acid sequence of SEQ ID NO: 3 or a sequence having one, two, three, or four amino acid substitutions (e.g., conservative amino acid substitutions); and/or

a light chain variable region (VL) comprising a light chain complementarity determining region 1 (VLCDR1) amino acid sequence of SEQ ID NO: 4 or a sequence having one, two, three, or four amino acid substitutions (e.g., conservative amino acid substitutions), a light chain complementarity determining region 2 (VLCDR2) amino acid sequence of SEQ ID NO: 5 or a sequence having one, two, three, or four amino acid substitutions (e.g., conservative amino acid substitutions), and a light chain complementarity determining region 3 (VLCDR3) amino acid sequence of SEQ ID NO: 6 or a sequence having one, two, three, or four amino acid substitutions (e.g., conservative amino acid substitutions).

In some embodiments of aspect A1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 2 or a sequence having one, two, or three amino acid substitutions, and a heavy chain complementarity determining region 3 (VHCDR3) amino acid sequence of SEQ ID NO: 3; and/or

a light chain variable region (VL) comprising a light chain complementarity determining region 1 (VLCDR1) amino acid sequence of SEQ ID NO: 4 or a sequence having one, or two amino acid substitutions, a light chain complementarity determining region 2 (VLCDR2) amino acid sequence of SEQ ID NO: 5, and a light chain complementarity determining region 3 (VLCDR3) amino acid sequence of SEQ ID NO: 6 or a sequence having one, two, three, or four amino acid substitutions.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 51, and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 52, a light chain complementarity determining region 2 (VLCDR2) amino acid sequence of SEQ ID NO: 5, and a light chain complementarity determining region 3 (VLCDR3) amino acid sequence of SEQ ID NO: 6.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 45, and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 46, a light chain complementarity determining region 2 (VLCDR2) amino acid sequence of SEQ ID NO: 5, and a light chain complementarity determining region 3 (VLCDR3) amino acid sequence of SEQ ID NO: 6.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 2, and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 4, a light chain complementarity determining region 2 (VLCDR2) amino acid sequence of SEQ ID NO: 5, and a light chain complementarity determining region 3 (VLCDR3) amino acid sequence of SEQ ID NO: 6.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 51 and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 52, a light chain complementarity determining region 2 (VLCDR2) amino acid sequence of SEQ ID NO: 5, and a light chain complementarity determining region 3 (VLCDR3) amino acid sequence of SEQ ID NO: 6;

wherein one, two, three, four, five, six, seven, or eight amino acids within a CDR have been inserted, deleted or substituted.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 45, and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 46, a light chain complementarity determining region 2 (VLCDR2) amino acid sequence of SEQ ID NO: 5, and a light chain complementarity determining region 3 (VLCDR3) amino acid sequence of SEQ ID NO: 6;

wherein one, two, three, four, five, six, seven, or eight amino acids within a CDR have been inserted, deleted or substituted.

In some embodiments of aspect A1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 2, and a heavy chain complementarity determining region 3 (VHCDR3) amino acid sequence of SEQ ID NO: 3; and

a light chain variable region (VL) comprising a light chain complementarity determining region 1 (VLCDR1) amino acid sequence of SEQ ID NO: 4, a light chain complementarity determining region 2 (VLCDR2) amino acid sequence of SEQ ID NO: 5, and a light chain complementarity determining region 3 (VLCDR3) amino acid sequence of SEQ ID NO: 6;

wherein one, two, three, four, five, six, seven, or eight amino acids within a CDR have been inserted, deleted or substituted.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 47, or an amino acid sequence having at least about 85%, 90%, 95%, or 99% sequence identity to SEQ ID NO: 47.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 7, or an amino acid sequence having at least about 85%, 90%, 95%, or 99% sequence identity to SEQ ID NO: 7.

In some embodiments of aspect A1, the anti-Siglec-9 antibody molecule or an anti-Siglec-9 binding fragment thereof a light chain variable region (VL) comprising the amino acid sequence of SEQ ID NO: 48, or an amino acid sequence having at least about 85%, 90%, 95%, or 99% sequence identity to SEQ ID NO: 48.

In some embodiments of aspect A1, the anti-Siglec-9 antibody molecule or an anti-Siglec-9 binding fragment thereof a light chain variable region (VL) comprising the amino acid sequence of SEQ ID NO: 8, or an amino acid sequence having at least about 85%, 90%, 95%, or 99% sequence identity to SEQ ID NO: 8.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 47, or an amino acid sequence having at least about 85%, 90%, 95%, or 99% sequence identity to SEQ ID NO: 47; and a light chain variable region (VL) comprising the amino acid sequence of SEQ ID NO: 48, or an amino acid sequence having at least about 85%, 90%, 95%, or 99% sequence identity to SEQ ID NO: 48.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 7, or an amino acid sequence having at least about 85%, 90%, 95%, or 99% sequence identity to SEQ ID NO: 7; and a light chain variable region (VL) comprising the amino acid sequence of SEQ ID NO: 8, or an amino acid sequence having at least about 85%, 90%, 95%, or 99% sequence identity to SEQ ID NO: 8.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 47, and a light chain variable region (VL) comprising the amino acid sequence of SEQ ID NO: 48.

In some embodiments of aspect A1, the anti-Siglec-9 antibody molecule or an anti-Siglec-9 binding fragment thereof comprises a heavy chain variable region (VH) comprising the amino acid sequence of SEQ ID NO: 7, and a light chain variable region (VL) comprising the amino acid sequence of SEQ ID NO: 8.

In some embodiments of aspect A1, the anti-Siglec-9 antibody molecule or an anti-Siglec-9 binding fragment thereof is a humanized anti-Siglec-9 antibody molecule or an anti-Siglec-9 binding fragment thereof.

### Functional Properties

In some embodiments of aspect A1, the anti-Siglec-9 antibody molecule or an anti-Siglec-9 binding fragment thereof comprises one or more (e.g., 2, 3, 4, 5, 6, 7, 8 or 9) of the following properties:

(i) binds to human Siglec-9 with a EC50 of less than about 10 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM e.g., when the antibody molecule or binding fragment thereof is tested as a bivalent molecule using ELISA, e.g., as described in Example 3;

(ii) binds to human Siglec-9 with a dissociation constant (KD) of less than about 10 nM, 1 nM, 0.9 nM, 0.8 nM, 0.7 nM, 0.6 nM, 0.5 nM, 0.4 nM, 0.3 nM, 0.2 nM or 0.1 nM, when the antibody molecule or binding fragment thereof is tested as a bivalent molecule using surface plasmon resonance, e.g. Carterra LSA, e.g., as described in Example 4;

(iii) binds to human CD14+ monocytes with a EC50 of less than about 10 nM, 1 nM, 0.9 nM, 0.8 nM, 0.7 nM, 0.6 nM, 0.5 nM, 0.4 nM, 0.3 nM, 0.2 nM or 0.1 nM, e.g., when the antibody molecule or binding fragment thereof is tested as a bivalent molecule using flow cytometer, e.g., as described in Example 5;

(vi) inhibits interaction between Siglec-9 and one or more Siglec-9 ligands, e.g., inhibits the binding of sialic-acid expressing A549 tumour cells to Siglec-9, e.g., when the antibody molecule or binding fragment thereof is tested as a bivalent molecule using ELISA, e.g., as described in Example 6;

(v) increases proliferation of anti-CD3/anti-CD28 stimulated CD8+ T cells, e.g., when the antibody molecule or binding fragment thereof is tested as a bivalent molecule in a T cell activation assay using Cell Trace Violet and flow cytometer, e.g., as described in Example 7;

(vi) upregulates activation markers CD69 and CD25 of anti-CD3/anti-CD28 stimulated CD8+ T cells, e.g., when the antibody molecule or binding fragment thereof is tested as a bivalent molecule in a T cell activation assay using staining and flow cytometer, e.g., as described in Example 7;

(vii) increases proliferation of anti-CD3/anti-CD28 stimulated CD4+ T cells, e.g., when the antibody molecule or binding fragment thereof is tested as a bivalent molecule in a T cell activation assay using Cell Trace Violet and flow cytometer, e.g., as described in Example 7;

(viii) upregulates activation markers CD69 and CD25 of anti-CD3/anti-CD28 stimulated CD4+ T cells, e.g., when the antibody molecule or binding fragment thereof is tested as a bivalent molecule in a T cell activation assay using staining and flow cytometer, e.g., as described in Example 7; or

(ix) increases NK cell-mediated killing activity, e.g., increases killing of K562 cells by NK92 cells overexpressing Siglec-9, e.g., when the antibody molecule or binding fragment thereof is tested as a bivalent molecule using Calcein-AM labeled K562 cells, e.g., as described in Example 8.

In one aspect A2, the present disclosure relates to an antibody molecule or a binding fragment thereof that competes for binding to human Siglec-9 with an antibody molecule or a binding fragment thereof described herein. In some embodiments of aspect A2, the present disclosure relates to an antibody molecule or a binding fragment thereof that competes for binding to human Siglec-9 with an antibody molecule or a binding fragment thereof that comprises a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 2, and a heavy chain complementarity determining region 3 (VHCDR3) amino acid sequence of SEQ ID NO: 3; and a light chain variable region (VL) comprising a light chain complementarity determining region 1 (VLCDR1) amino acid sequence of SEQ ID NO: 4, a light chain complementarity determining region 2 (VLCDR2) amino acid sequence of SEQ ID NO: 5, and a light chain complementarity determining region 3 (VLCDR3) amino acid sequence of SEQ ID NO: 6. In some embodiments of aspect A2, the present disclosure relates to an antibody molecule or a binding fragment thereof that competes for binding to human Siglec-9 with an antibody molecule or a binding fragment thereof that comprises a heavy chain variable region (VH) comprising the amino acid sequence of SEQ ID NO: 7, and a light chain variable region (VL) comprising the amino acid sequence of SEQ ID NO: 8

In one aspect A3, the present disclosure relates to a pharmaceutical composition comprising the antibody molecule or binding fragment thereof described herein and a pharmaceutically acceptable carrier, excipient or stabilizer.

In one aspect A4, the present disclosure relates to anti-Siglec-9 antibody molecules or binding fragments thereof described herein, or pharmaceutical compositions comprising anti-Siglec-9 antibody molecules or binding fragments thereof described herein, for use in the treatment of a disease selected from the group consisting of cancer, acute and chronic hepatitis B. In some embodiments of aspect A4, the cancer is selected from the group consisting of non-small cell lung cancer, colorectal cancer, breast cancer, epithelial ovarian cancer, hepatocellular carcinoma and prostate cancer.

In one aspect A5, the present disclosure relates to a nucleic acid encoding the antibody heavy and/or light chain variable region of the antibody molecule or binding fragment thereof described herein.

In one aspect A6, the present disclosure relates to an expression vector comprising the nucleic acid described herein.

In one aspect A7, the present disclosure relates to a host cell comprising the nucleic acid described herein or the expression vector described herein.

In one aspect A8, the present disclosure relates to a method of producing an antibody molecule, the method comprising culturing the host cell described herein under conditions suitable for gene expression.

Aspect B

In one aspect B1, the present disclosure relates to an anti-Siglec-9 antibody molecule or an anti-Siglec-9 binding fragment thereof.

### Structural Properties

In some embodiments of aspect B1, the present disclosure relates to an anti-Siglec-9 antibody molecule or an anti-Siglec-9 binding fragment thereof, wherein the antibody molecule or binding fragment comprises at least one, two, three, four, five or six complementarity determining regions (CDRs) (or collectively all of the CDRs) from a heavy chain variable region (VH) and/or a light chain variable region (VL) comprising an amino acid sequence shown in Table 2, wherein one or more of the CDRs (or collectively all of the CDRs) may have one, two, three, four, five, six or more changes, e.g., amino acid substitutions (e.g., conservative amino acid substitutions), insertions or deletions, relative to an amino acid sequence shown in Table 2.

In some embodiments of aspect B1, the present disclosure relates to an anti-Siglec-9 antibody molecule or an anti-Siglec-9 binding fragment thereof, comprising:

a heavy chain variable region (VH) comprising one, two, or three of: a heavy chain complementarity determining region 1 (VHCDR1) amino acid sequence of SEQ ID NO: 30 or a sequence having one, two, three, or four amino acid substitutions (e.g., conservative amino acid substitutions), insertions or deletions, a heavy chain complementarity determining region 2 (VHCDR2) amino acid sequence of SEQ ID NO: 31 or a sequence having one, two, three, or four amino acid substitutions (e.g., conservative amino acid substitutions), insertions or deletions, and a heavy chain complementarity determining region 3 (VHCDR3) amino acid sequence of SEQ ID NO: 32 or a sequence having one, two, three, or four amino acid substitutions (e.g., conservative amino acid substitutions), insertions or deletions; and/or a light chain variable region (VL) comprising one, two, or three of: a light chain complementarity determining region 1 (VLCDR1) amino acid sequence of SEQ ID NO: 33 or a sequence having one, two, three, or four amino acid substitutions (e.g., conservative amino acid substitutions), insertions or deletions, a light chain complementarity determining region 2 (VLCDR2) amino acid sequence of SEQ ID NO: 34 or a sequence having one, two, three, or four amino acid substitutions (e.g., conservative amino acid substitutions), insertions or deletions, and a light chain complementarity determining region 3 (VLCDR3) amino acid sequence of SEQ ID NO: 35 or a sequence having one, two, three, or four amino acid substitutions (e.g., conservative amino acid substitutions), insertions or deletions.

In some embodiments of aspect B1, the present disclosure relates to an anti-Siglec-9 antibody molecule or an anti-Siglec-9 binding fragment thereof comprising:

a heavy chain variable region (VH) comprising a heavy chain complementarity determining region 3 (VHCDR3) amino acid sequence of SEQ ID NO: 32, or a sequence having one, two, three, or four amino acid substitutions (e.g., conservative amino acid substitutions); and/or

a light chain variable region (VL) comprising a light chain complementarity determining region 3 (VLCDR3) amino acid sequence of SEQ ID NO: 35, or a sequence having one, two, three, or four amino acid substitutions (e.g., conservative amino acid substitutions).

In some embodiments of aspect B1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 30 or a sequence having one, two, three, or four amino acid substitutions (e.g., conservative amino acid substitutions), a heavy chain complementarity determining region 2 (VHCDR2) amino acid sequence of SEQ ID NO: 31 or a sequence having one, two, three, or four amino acid substitutions (e.g., conservative amino acid substitutions), and a heavy chain complementarity determining region 3 (VHCDR3) amino acid sequence of SEQ ID NO: 32 or a sequence having one, two, three, or four amino acid substitutions (e.g., conservative amino acid substitutions); and/or

a light chain variable region (VL) comprising a light chain complementarity determining region 1 (VLCDR1) amino acid sequence of SEQ ID NO: 33 or a sequence having one, two, three, or four amino acid substitutions (e.g., conservative amino acid substitutions), a light chain complementarity determining region 2 (VLCDR2) amino acid sequence of SEQ ID NO: 34 or a sequence having one, two, three, or four amino acid substitutions (e.g., conservative amino acid substitutions), and a light chain complementarity determining region 3 (VLCDR3) amino acid sequence of SEQ ID NO: 35 or a sequence having one, two, three, or four amino acid substitutions (e.g., conservative amino acid substitutions).

In some embodiments of aspect B1, the present disclosure relates to an anti-Siglec-9 antibody molecule or an anti-Siglec-9 binding fragment thereof, comprising:

a heavy chain variable region (VH) comprising a heavy chain complementarity determining region 1 (VHCDR1) amino acid sequence of SEQ ID NO: 30, a heavy chain complementarity determining region 2 (VHCDR2) amino acid sequence of SEQ ID NO: 31 or a sequence having one, two, or three amino acid substitutions, and a heavy chain complementarity determining region 3 (VHCDR3) amino acid sequence of SEQ ID NO: 32; and/or

a light chain variable region (VL) comprising a light chain complementarity determining region 1 (VLCDR1) amino acid sequence of SEQ ID NO: 33 or a sequence having one, or two amino acid substitutions, a light chain complementarity determining region 2 (VLCDR2) amino acid sequence of SEQ ID NO: 34, and a light chain complementarity determining region 3 (VLCDR3) amino acid sequence of

SEQ ID NO: 35 or a sequence having one, two, three, or four amino acid substitutions.

In some embodiments of aspect B1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 30, a heavy chain complementarity determining region 2 (VHCDR2) amino acid sequence of SEQ ID NO: 31, and a heavy chain complementarity determining region 3 (VHCDR3) amino acid sequence of SEQ ID NO: 32; and

a light chain variable region (VL) comprising a light chain complementarity determining region 1 (VLCDR1) amino acid sequence of SEQ ID NO: 33, a light chain complementarity determining region 2 (VLCDR2) amino acid sequence of SEQ ID NO: 34, and a light chain complementarity determining region 3 (VLCDR3) amino acid sequence of SEQ ID NO: 35.

In some embodiments of aspect B1, the anti-Siglec-9 antibody molecule or an anti-Siglec-9 binding fragment thereof comprises:

a heavy chain variable region (VH) comprising a heavy chain complementarity determining region 1 (VHCDR1) amino acid sequence of SEQ ID NO: 30, a heavy chain complementarity determining region 2 (VHCDR2) amino acid sequence of SEQ ID NO: 31, and a heavy chain complementarity determining region 3 (VHCDR3) amino acid sequence of SEQ ID NO: 32; and

a light chain variable region (VL) comprising a light chain complementarity determining region 1 (VLCDR1) amino acid sequence of SEQ ID NO: 33, a light chain complementarity determining region 2 (VLCDR2) amino acid sequence of SEQ ID NO: 34, and a light chain complementarity determining region 3 (VLCDR3) amino acid sequence of SEQ ID NO: 35;

wherein one, two, three, four, five, six, seven, or eight amino acids within a CDR have been inserted, deleted or substituted.

In some embodiments of aspect B1, the anti-Siglec-9 antibody molecule or an anti-Siglec-9 binding fragment thereof comprises a heavy chain variable region (VH) comprising the amino acid sequence of SEQ ID NO: 36, or an amino acid sequence having at least about 85%, 90%, 95%, or 99% sequence identity to SEQ ID NO: 36.

In some embodiments of aspect B1, the anti-Siglec-9 antibody molecule or an anti-Siglec-9 binding fragment thereof a light chain variable region (VL) comprising the amino acid sequence of SEQ ID NO: 37, or an amino acid sequence having at least about 85%, 90%, 95%, or 99% sequence identity to SEQ ID NO: 37.

In some embodiments of aspect B1, the anti-Siglec-9 antibody molecule or an anti-Siglec-9 binding fragment thereof comprises a heavy chain variable region (VH) comprising the amino acid sequence of SEQ ID NO: 36, or an amino acid sequence having at least about 85%, 90%, 95%, or 99% sequence identity to SEQ ID NO: 36; and a light chain variable region (VL) comprising the amino acid sequence of SEQ ID NO: 37, or an amino acid sequence having at least about 85%, 90%, 95%, or 99% sequence identity to SEQ ID NO: 37.

In some embodiments of aspect B1, the anti-Siglec-9 antibody molecule or an anti-Siglec-9 binding fragment thereof comprises a heavy chain variable region (VH) comprising the amino acid sequence of SEQ ID NO: 36, and a light chain variable region (VL) comprising the amino acid sequence of SEQ ID NO: 37.

In some embodiments of aspect B1, the anti-Siglec-9 antibody molecule or an anti-Siglec-9 binding fragment thereof is a humanized anti-Siglec-9 antibody molecule or an anti-Siglec-9 binding fragment thereof.

### Functional Properties

In some embodiments of aspect B1, the anti-Siglec-9 antibody molecule or an anti-Siglec-9 binding fragment thereof comprises one or more (e.g., 2, 3, 4, 5, 6, 7, 8, 9 or 10) of the following properties:

(i) binds to human Siglec-9 with a EC50 of less than about 10 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM, e.g., when the antibody molecule or binding fragment thereof is tested as a bivalent molecule using ELISA, e.g., as described in Example 3;

(ii) binds to human Siglec-9 with a dissociation constant (KD) of less than about 10 nM, 1 nM, 0.9 nM, 0.8 nM, 0.7 nM, 0.6 nM, 0.5 nM, 0.4 nM, 0.3 nM, 0.2 nM or 0.1 nM, when the antibody molecule or binding fragment thereof is tested as a bivalent molecule using surface plasmon resonance, e.g. Carterra LSA, e.g., as described in Example 4;

(iii) binds to human CD14+ monocytes with a EC50 of less than about 10 nM, 1 nM, 0.9 nM, 0.8 nM, 0.7 nM, 0.6 nM, 0.5 nM, 0.4 nM, 0.3 nM, 0.2 nM or 0.1 nM, e.g., when the antibody molecule or binding fragment thereof is tested as a bivalent molecule using flow cytometer, e.g., as described in Example 5;

(vi) inhibits interaction between Siglec-9 and one or more Siglec-9 ligands, e.g., inhibits the binding of sialic-acid expressing A549 tumour cells to Siglec-9, e.g., when the antibody molecule or binding fragment thereof is tested as a bivalent molecule using ELISA, e.g., as described in Example 6;

(v) increases proliferation of anti-CD3/anti-CD28 stimulated CD8+ T cells, e.g., when the antibody molecule or binding fragment thereof is tested as a bivalent molecule in a T cell activation assay using Cell Trace Violet and flow cytometer, e.g., as described in Example 7;

(vi) upregulates activation markers CD69 and CD25 of anti-CD3/anti-CD28 stimulated CD8+ T cells, e.g., when the antibody molecule or binding fragment thereof is tested as a bivalent molecule in a T cell activation assay using staining and flow cytometer, e.g., as described in Example 7;

(vii) increases proliferation of anti-CD3/anti-CD28 stimulated CD4+ T cells, e.g., when the antibody molecule or binding fragment thereof is tested as a bivalent molecule in a T cell activation assay using Cell Trace Violet and flow cytometer, e.g., as described in Example 7;

(viii) upregulates activation markers CD69 and CD25 of anti-CD3/anti-CD28 stimulated CD4+ T cells, e.g., when the antibody molecule or binding fragment thereof is tested as a bivalent molecule in a T cell activation assay using staining and flow cytometer, e.g., as described in Example 7;

(ix) increases NK cell-mediated killing activity, e.g., increases killing of K562 cells by NK92 cells overexpressing Siglec-9, e.g., when the antibody molecule or binding fragment thereof is tested as a bivalent molecule using Calcein-AM labeled K562 cells, e.g., as described in Example 8; or

(x) binds to human Siglec-7.

In one aspect B2, the present disclosure relates to an antibody molecule or a binding fragment thereof that competes for binding to human Siglec-9 with an antibody molecule or a binding fragment thereof described herein. In some embodiments of aspect B2, the present disclosure relates to an antibody molecule or a binding fragment thereof that competes for binding to human Siglec-9 with an antibody molecule or a binding fragment thereof that comprises a heavy chain variable region (VH) comprising a heavy chain complementarity determining region 1 (VHCDR1) amino acid sequence of SEQ ID NO: 30, a heavy chain complementarity determining region 2 (VHCDR2) amino acid sequence of SEQ ID NO: 31, and a heavy chain complementarity determining region 3 (VHCDR3) amino acid sequence of SEQ ID NO: 32; and a light chain variable region (VL) comprising a light chain complementarity determining region 1 (VLCDR1) amino acid sequence of SEQ ID NO: 33, a light chain complementarity determining region 2 (VLCDR2) amino acid sequence of SEQ ID NO: 34, and a light chain complementarity determining region 3 (VLCDR3) amino acid sequence of SEQ ID NO: 35. In some embodiments of aspect B2, the present disclosure relates to an antibody molecule or a binding fragment thereof that competes for binding to human Siglec-9 with an antibody molecule or a binding fragment thereof that comprises a heavy chain variable region (VH) comprising the amino acid sequence of SEQ ID NO: 36, and a light chain variable region (VL) comprising the amino acid sequence of SEQ ID NO: 37

In one aspect B3, the present disclosure relates to a pharmaceutical composition comprising the antibody molecule or binding fragment thereof described herein and a pharmaceutically acceptable carrier, excipient or stabilizer.

In one aspect B4, the present disclosure relates to anti-Siglec-9 antibody molecules or binding fragments thereof described herein, or pharmaceutical compositions comprising anti-Siglec-9 antibody molecules or binding fragments thereof described herein, for use in the treatment of a disease selected from the group consisting of cancer, acute and chronic hepatitis B. In some embodiments, the cancer is selected from the group consisting of non-small cell lung cancer, colorectal cancer, breast cancer, epithelial ovarian cancer, hepatocellular carcinoma and prostate cancer.

In one aspect B5, the present disclosure relates to a nucleic acid encoding the antibody heavy and/or light chain variable region of the antibody molecule or binding fragment thereof described herein.

In one aspect B6, the present disclosure relates to an expression vector comprising the nucleic acid described herein.

In one aspect B7, the present disclosure relates to a host cell comprising the nucleic acid described herein or the expression vector described herein.

In one aspect B8, the present disclosure relates to a method of producing an antibody molecule, the method comprising culturing the host cell described herein under conditions suitable for gene expression.

### BRIEF DESCRIPTION OF THE DRAWINGS

FIG. 1: Reactivity of monoclonal antibodies (68D4 and 224B1; comparative monoclonal antibodies mAbA and 5C6 & negative control 53C3) towards Siglec-5, Siglec-7, Siglec-8, Siglec-15 and Siglec-9 as analysed by ELISA. Binding is determined by measurement of the accumulated coloured product at 450 nm after stopping the reaction (n=3).

FIG. 2: Binding curves of monoclonal antibodies (68D4 and 224B1; comparative monoclonal antibodies mAbA and 5C6 & negative control 53C3) towards Siglec-9 as analysed by ELISA. Binding of the monoclonal antibody to Siglec-9, as determined by measurement of the accumulated coloured product at 450 nm after stopping the reaction, is plotted against the concentration of the monoclonal antibody under evaluation. EC50 values as determined by three-parameter analysis in GraphPad Prism (GraphPad Software) are given in the bottom right corner of the graphs (n=3).

FIG. 3: Binding curves of monoclonal antibodies (68D4 and 224B1; comparative monoclonal antibodies mAbA and 5C6 & negative control 53C3) towards Siglec-9 displayed on human CD14+ monocytes as analysed by flow cytometry. The mean fluorescence signal of the secondary antibody bound to the CD14+ population is plotted against the concentration of the monoclonal antibody under evaluation. EC50 values as determined by three-parameter analysis in GraphPad Prism (GraphPad Software) are given in the bottom right corner of the graphs.

FIG. 4: Titration of monoclonal antibodies (68D4 and 224B1; comparative monoclonal antibodies mAbA and 5C6 & negative control 53C3) to determine blocking the binding of sialic acid-displaying A549 tumour cells to Siglec-9. Tumour cells were fluorescently-loaded with Calcein-AM in advance to the binding experiment and binding was determined by the released fluorescence upon cell lysis. Fluorescence released in the absence of antibody was set to 100% binding. Data was fitted by three-parameter analysis in GraphPad Prism (GraphPad Software); n=3.

FIG. 5: Activation assay of CD8+ T cells. Titration of the monoclonal antibodies (68D4 and 224B1; comparative monoclonal antibodies mAbA and 5C6 & negative control 53C3) and its effect on the proliferation (A), upregulation of the activation markers CD69 (B) and CD25 (C) of CD8+ T cells (co-stimulated by anti-CD3 and anti-CD28 antibodies) originating from a Siglec-9 transgenic mouse model. Proliferation and levels of the activation markers on CD8+ T cells were analysed by flow cytometry.

FIG. 6: Activation assay of CD4+ T cells. Titration of the monoclonal antibodies (68D4 and 224B1; comparative monoclonal antibodies mAbA and 5C6 & negative control 53C3) and its effect on the proliferation (A), upregulation of the activation markers CD69 (B) and CD25 (C) of CD4+ T cells (co-stimulated by anti-CD3 and anti-CD28 antibodies) originating from a Siglec-9 transgenic mouse model. Proliferation and levels of the activation markers on CD4+ T cells were analysed by flow cytometry.

FIG. 7: Titration of monoclonal antibodies (68D4 and 224B1; comparative monoclonal antibodies mAbA and 5C6 & negative control 53C3) to determine the killing of Calcein-AM labeled K562 cells by NK92 cells. Killing was determined by the released fluorescence upon cell lysis of K562 cells. Specific Lysis was calculated as: (Lysis test well−spontaneous lysis)/(maximal lysis−spontaneous lysis)×100%. Fluorescence released upon addition of the detergent Triton X-100 and incubation of K562 cells alone was used to determine maximal lysis and spontaneous lysis, respectively. Data was analysed by one site (total) analysis in GraphPad Prism (GraphPad Software); n=3.

FIG. 8: Humanization of rabbit antibody 68D4. Alignments are shown of the light chain variable domain of human IgG 263E11 (SEQ ID NO: 54), human germline sequences KV1-5/J4 (SEQ ID NO: 55 and SEQ ID NO: 56), rb 68D4 (SEQ ID NO: 8) and the resulting graft 68D4-hum (SEQ ID NO: 48); and the heavy chain variable domain of human IgG 263E11 (SEQ ID NO: 53), human germline sequences VH3-23/J4 (SEQ ID NO: 57 and SEQ ID NO: 58), rb 68D4 (SEQ ID NO: 7) and the resulting graft 68D4-hum (SEQ ID NO: 47).

FIG. 9: Binding curves of monoclonal antibodies (rabbit 68D4; humanized 68D4 and comparative monoclonal antibody mAbA) towards Siglec-9 as analysed by ELISA. Binding of the monoclonal antibody to Siglec-9, as determined by measurement of the accumulated coloured product at 450 nm after stopping the reaction, is plotted against the concentration of the monoclonal antibody under evaluation.

### DETAILED DESCRIPTION

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term indicates deviation from the indicated numerical value of ±20%, preferably ±10%, and more preferably of ±5%.

Technical terms are used by their common sense. If a specific meaning is conveyed to certain terms, definitions of terms will be given in the following in the context of which the terms are used.

Human Siglec-7 (NCBI Reference Sequence:
Q9Y286.1) has the following amino acid
sequence (SEQ ID NO: 60):
MLLLLLLPLLWGRERVEGQKSNRKDYSLTMQSSVTVQEGMCVHVRCSFSY
PVDSQTDSDPVHGYWFRAGNDISWKAPVATNNPAWAVQEETRDRFHLLGD
PQTKNCTLSIRDARMSDAGRYFFRMEKGNIKWNYKYDQLSVNVTALTHRP
NILIPGTLESGCFQNLTCSVPWACEQGTPPMISWMGTSVSPLHPSTTRSS
VLTLIPQPQHHGTSLTCQVTLPGAGVTTNRTIQLNVSYPPQNLTVTVFQG
EGTASTALGNSSSLSVLEGQSLRLVCAVDSNPPARLSWTWRSLTLYPSQP
SNPLVLELQVHLGDEGEFTCRAQNSLGSQHVSLNLSLQQEYTGKMRPVSG
VLLGAVGGAGATALVFLSFCVIFIWRSCRKKSARPAADVGDIGMKDANTI
RGSASQGNLTESWADDNPRHHGLAAHSSGEEREIQYAPLSFHKGEPQDLS
GQEATNNEYSEIKIPK
Human Siglec-9 (NCBI Reference Sequence:
NP_055256.1) is a member of the CD33-related
Siglec family and also referred to as Sialic
acid-binding Ig-like lectin 9, CD329, CDw329,
FOAP-9, and OBBP-LIKE. Human Siglec-9 comprises
463 amino acids, having the following amino
acid sequence (SEQ ID NO: 9):
MLLLLLPLLW GRERAEGQTS KLLTMQSSVT VQEGLCVHVP
CSFSYPSHGW IYPGPVVHGY WFREGANTDQ DAPVATNNPA
RAVWEETRDR FHLLGDPHTK NCTLSIRDAR RSDAGRYFFR
MEKGSIKWNY KHHRLSVNVT ALTHRPNILI PGTLESGCPQ
NLTCSVPWAC EQGTPPMISW IGTSVSPLDP STTRSSVLTL
IPQPQDHGTS LTCQVTFPGA SVTTNKTVHL NVSYPPQNLT
MTVFQGDGTV STVLGNGSSL SLPEGQSLRL VCAVDAVDSN
PPARLSLSWR GLTLCPSQPS NPGVLELPWV HLRDAAEFTC
RAQNPLGSQQ VYLNVSLQSK ATSGVTQGVV GGAGATALVF
LSFCVIFVVV RSCRKKSARP AAGVGDTGIE DANAVRGSAS
QGPLTEPWAE DSPPDQPPPA SARSSVGEGE LQYASLSFQM
VKPWDSRGQE ATDTEYSEIK IHR

Siglec-9 is an inhibitory immune receptor expressed in the tumor microenvironment by cells of the adaptive and innate immune system (e.g. NK cells, CD8+- and CD4+ T cells, and macrophages). Inhibitory signaling through this receptor was shown to be triggered by the expression of ligands upregulated on tumor cells and tumor stroma (Laubli et al., Proc Natl Acad Sci USA. 2014 Sep. 30; 111(39):14211-6). Importantly, CRC (colorectal cancer) and NSCLC (non-small cell lung cancer) cancer patient's CD8+ and CD4+ tumor infiltrating lymphocytes (TILs) show upregulation of Siglec-9, particularly in those TILs that express high levels of PD-1 (Stanczak et al., J Clin Invest. 2018 Nov. 1; 128(11):4912-4923). In addition, blocking of Siglec-9 was suggested as a potential way to control hepatitis B virus replication (Zhao et al., Front Immunol. 2018 May 30; 9:1124).

Certain aspects of the present disclosure are based, at least in part, on the identification of anti-Siglec-9 antibody molecules or binding fragments thereof that

inhibit interaction between Siglec-9 and one or more Siglec-9 ligands; and/or

increase proliferation of anti-CD3/anti-CD28 stimulated CD8+ T cells; and/or

upregulate activation markers CD69 and CD25 of anti-CD3/anti-CD28 stimulated CD8+ T cells; and/or

increase proliferation of anti-CD3/anti-CD28 stimulated CD4+ T cells; and/or

upregulate activation markers CD69 and CD25 of anti-CD3/anti-CD28 stimulated CD4+ T cells; and/or

increase NK cell-mediated killing activity.

As mentioned above, the present disclosure considers anti-Siglec-9 antibody molecules or binding fragments thereof. A full-length antibody includes a constant domain and a variable domain. The constant region need not be present in an antigen-binding fragment of an antibody.

Binding fragments may thus include portions of an intact full-length antibody, such as an antigen binding or variable region of the complete antibody. Examples of antibody fragments include Fab, F(ab′)2, Id and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); minibodies; chelating recombinant antibodies; tribodies or bibodies; intrabodies; nanobodies; small modular immunopharmaceuticals (SMTP), binding-domain immunoglobulin fusion proteins; camelized antibodies; VHH containing antibodies; and any other polypeptides formed from antibody fragments. The skilled person is aware that the antigen-binding function of an antibody can be performed by fragments of a full-length antibody.

Humanized antibodies are also within the present disclosure.

Disclosed herein are polypeptides having the sequences specified, or sequences substantially identical or similar thereto, e.g. sequences having at least about 85%, 90%, 95%, or 99% sequence identity to the sequence specified.

The determination of percent identity between two sequences is preferably accomplished using the mathematical algorithm of Karlin and Altschul (1993) Proc. Natl. Acad. Sci USA 90: 5873-5877. Such an algorithm is incorporated into the BLASTp (Protein BLAST) program of Altschul et al. (1990) J. Mol. Biol. 215: 403-410 available at NCBI (https://blast.ncbi.nlm.nih.gov/). The determination of percent identity may be performed with the standard parameters of the BLASTp program. For the general parameters, the “Max Target Sequences” box may be set to 100, the “Short queries” box may be ticked, the “Expect threshold” box may be set to 10, the “Word Size” box may be set to “3” and the “Max matches in a query range” may be set to “0”. For the scoring parameters the “Matrix” box may be set to “BLOSU M62”, the “Gap Costs” Box may be set to “Existence: 11 Extension: 1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

According to the disclosure, a “conservative amino acid substitution” is an amino acid substitution in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As mentioned, the disclosure also relates in some embodiments to a nucleic acid encoding antibody molecules or binding fragments thereof, vectors comprising such nucleic acids and host cells comprising such nucleic acids or vectors.

The antibody molecules or binding fragments thereof may be encoded by a single nucleic acid (e.g., a single nucleic acid comprising nucleotide sequences that encode the light and heavy chain polypeptides of the antibody), or by two or more separate nucleic acids, each of which encode a different part of the antibody molecule or antibody fragment. The nucleic acids may be DNA, cDNA, RNA and the like.

The nucleic acids described herein can be inserted into vectors. A “vector” is any molecule or composition that has the ability to carry a nucleic acid sequence into a suitable cell where synthesis of the encoded polypeptide can take place.

The present disclosure in some aspects further provides a host cell (e.g., an isolated or purified cell) comprising a nucleic acid or vector of the invention. The host cell can be any type of cell capable of being transformed with the nucleic acid or vector of the invention so as to produce a polypeptide encoded thereby.

The anti-Siglec-9 antibody molecules or anti-Siglec-9 binding fragments thereof can be formulated in compositions, especially pharmaceutical compositions. Such compositions comprise a therapeutically effective amount of an antibody or binding fragment thereof in admixture with a pharmaceutically acceptable carrier, excipient or stabilizer.

Further, the anti-Siglec-9 antibody molecules or anti-Siglec-9 binding fragments thereof and the pharmaceutical compositions as described herein can be administered in methods of treating patients with a disease selected from the group consisting of cancer, acute and chronic hepatitis B. This disease may be characterized by upregulation of one or more Siglec-9 ligands.

Treatment of non-small cell lung cancer, colorectal cancer, breast cancer, epithelial ovarian cancer, hepatocellular carcinoma and prostate cancer may be particularly effective when using the anti-Siglec-9 antibody molecules or anti-Siglec-9 binding fragments or pharmaceutical compositions as described herein.

Preferred embodiments of aspects B1 to B8 of the present invention relate to:

1. An anti-Siglec-9 antibody molecule or an anti-Siglec-9 binding fragment thereof comprising:

a heavy chain variable region (VH) comprising a heavy chain complementarity determining region 1 (VHCDR1) amino acid sequence of SEQ ID NO: 30 or a sequence having one, two, three, or four amino acid substitutions, a heavy chain complementarity determining region 2 (VHCDR2) amino acid sequence of SEQ ID NO: 31 or a sequence having one, two, three, or four amino acid substitutions, and a heavy chain complementarity determining region 3 (VHCDR3) amino acid sequence of SEQ ID NO: 32 or a sequence having one, two, three, or four amino acid substitutions; and

a light chain variable region (VL) comprising a light chain complementarity determining region 1 (VLCDR1) amino acid sequence of SEQ ID NO: 33 or a sequence having one, two, three, or four amino acid substitutions, a light chain complementarity determining region 2 (VLCDR2) amino acid sequence of SEQ ID NO: 34 or a sequence having one, two, three, or four amino acid substitutions, and a light chain complementarity determining region 3 (VLCDR3) amino acid sequence of SEQ ID NO: 35 or a sequence having one, two, three, or four amino acid substitutions.

2. The antibody molecule or binding fragment thereof of item 1, comprising:

a heavy chain variable region (VH) comprising a heavy chain complementarity determining region 1 (VHCDR1) amino acid sequence of SEQ ID NO: 30, a heavy chain complementarity determining region 2 (VHCDR2) amino acid sequence of SEQ ID NO: 31, and a heavy chain complementarity determining region 3 (VHCDR3) amino acid sequence of SEQ ID NO: 32; and

a light chain variable region (VL) comprising a light chain complementarity determining region 1 (VLCDR1) amino acid sequence of SEQ ID NO: 33, a light chain complementarity determining region 2 (VLCDR2) amino acid sequence of SEQ ID NO: 34, and a light chain complementarity determining region 3 (VLCDR3) amino acid sequence of SEQ ID NO: 35.

3. The antibody molecule or binding fragment thereof of items 1 or 2, comprising a heavy chain variable region (VH) comprising the amino acid sequence of SEQ ID NO: 36, or an amino acid sequence having at least about 85%, 90%, 95%, or 99% sequence identity to SEQ ID NO: 36.

4. The antibody molecule or binding fragment thereof of any one of items 1-3, comprising a light chain variable region (VL) comprising the amino acid sequence of SEQ ID NO: 37, or an amino acid sequence having at least about 85%, 90%, 95%, or 99% sequence identity to SEQ ID NO: 37.

5. The antibody molecule or binding fragment thereof of any one of items 1-4, comprising a heavy chain variable region (VH) comprising the amino acid sequence of SEQ ID NO: 36, and a light chain variable region (VL) comprising the amino acid sequence of SEQ ID NO: 37.

6. The antibody molecule or binding fragment thereof of any one of items 1-5, comprising one or more (e.g., 2, 3, 4, 5, 6, 7, 8, 9 or 10) of the following properties:

(i) binds to human Siglec-9 with a EC50 of less than about 10 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM, e.g., when the antibody molecule or binding fragment thereof is tested as a bivalent molecule using ELISA, e.g., as described in Example 3;

(ii) binds to human Siglec-9 with a dissociation constant (KD) of less than about 10 nM, 1 nM, 0.9 nM, 0.8 nM, 0.7 nM, 0.6 nM, 0.5 nM, 0.4 nM, 0.3 nM, 0.2 nM or 0.1 nM, when the antibody molecule or binding fragment thereof is tested as a bivalent molecule using surface plasmon resonance, e.g. Carterra LSA, e.g., as described in Example 4;

(iii) binds to human CD14+ monocytes with a EC50 of less than about 10 nM, 1 nM, 0.9 nM, 0.8 nM, 0.7 nM, 0.6 nM, 0.5 nM, 0.4 nM, 0.3 nM, 0.2 nM or 0.1 nM, e.g., when the antibody molecule or binding fragment thereof is tested as a bivalent molecule using flow cytometer, e.g., as described in Example 5;

(vi) inhibits interaction between Siglec-9 and one or more Siglec-9 ligands, e.g., inhibits the binding of sialic-acid expressing A549 tumour cells to Siglec-9, e.g., when the antibody molecule or binding fragment thereof is tested as a bivalent molecule using ELISA, e.g., as described in Example 6;

(v) increases proliferation of anti-CD3/anti-CD28 stimulated CD8+ T cells, e.g., when the antibody molecule or binding fragment thereof is tested as a bivalent molecule in a T cell activation assay using Cell Trace Violet and flow cytometer, e.g., as described in Example 7;

(vi) upregulates activation markers CD69 and CD25 of anti-CD3/anti-CD28 stimulated CD8+ T cells, e.g., when the antibody molecule or binding fragment thereof is tested as a bivalent molecule in a T cell activation assay using staining and flow cytometer, e.g., as described in Example 7;

(vii) increases proliferation of anti-CD3/anti-CD28 stimulated CD4+ T cells, e.g., when the antibody molecule or binding fragment thereof is tested as a bivalent molecule in a T cell activation assay using Cell Trace Violet and flow cytometer, e.g., as described in Example 7;

(viii) upregulates activation markers CD69 and CD25 of anti-CD3/anti-CD28 stimulated CD4+ T cells, e.g., when the antibody molecule or binding fragment thereof is tested as a bivalent molecule in a T cell activation assay using staining and flow cytometer, e.g., as described in Example 7;

(ix) increases NK cell-mediated killing activity, e.g., increases killing of K562 cells by N K92 cells overexpressing Siglec-9, e.g., when the antibody molecule or binding fragment thereof is tested as a bivalent molecule using Calcein-AM labeled K562 cells, e.g., as described in Example 8; or

(x) binds to human Siglec-7.

7. An antibody molecule or a binding fragment thereof that competes for binding to human Siglec-9 with the antibody molecule or binding fragment thereof of any one of items 1-6.

8. The antibody molecule or binding fragment thereof of item 7 that competes for binding to the same epitope of human Siglec-9 with the antibody molecule or binding fragment thereof of any one of items 1-6.

9. A pharmaceutical composition comprising the antibody molecule or binding fragment thereof of any one of items 1-8 and a pharmaceutically acceptable carrier, excipient or stabilizer.

10. An antibody molecule or binding fragment thereof according to any one of items 1-8 or a pharmaceutical composition according to item 9 for use in the treatment of a disease selected from the group consisting of cancer, acute and chronic hepatitis B.

11. An antibody molecule or binding fragment thereof according to any one of items 1-8 or a pharmaceutical composition according to item 9 for the use of item 10, wherein the cancer is selected from the group consisting of non-small cell lung cancer, colorectal cancer, breast cancer, epithelial ovarian cancer, hepatocellular carcinoma and prostate cancer.

12. A nucleic acid encoding the antibody heavy and/or light chain variable region of the antibody molecule or binding fragment thereof of any one of items 1-8.

13. An expression vector comprising the nucleic acid of item 12.

14. A host cell comprising the nucleic acid of item 12 or the expression vector of item 13.

15. A method of producing an antibody molecule, the method comprising culturing the host cell of item 14 under conditions suitable for gene expression.

### EXAMPLES

### Antibody Discovery

### Example 1a

NZW rabbits were immunized by DNA-immunization or by injection of Siglec-9-Fc-protein and antibody repertoire expression libraries were derived from lymph node and spleen B cells by cloning the immunoglobulin light chain and heavy chain variable region into an expression cassette providing the rabbit immunoglobulin constant heavy region and a transmembrane domain derived from human CD8. Screening of the antibody libraries was performed after transduction of the library in HEK 293T cells followed by antigen-specific sorting using fluorescently labeled Siglec-9. This sort yielded more than 1000 Siglec-9-specific-antibody-expressing cell clones which were further propagated for downstream analysis of antibody properties. Siglec-9-specific antibodies with high affinity and no cross-reactivity to Siglec-7 were then sub-cloned into expression vectors for soluble antibody expression and expressed after transient transfection in HEK 293F cells. Antibodies were then characterized in the various assays after purification based on protein G.

The Siglec-9 DNA sequence used for genetic
immunization had the following sequence
(codon-optimized compared to human sequence):
(SEQ ID NO: 10)
CCTGCAGGCCGCTAGCACCATGCTGCTGCTGCTGCTGCCCCTGCTGTGGG
GCCGCGAGCGCGCCGAGGGCCAGACCTCCAAGCTGCTGACCATGCAGTCC
TCCGTGACCGTGCAGGAGGGCCTGTGCGTGCACGTGCCCTGCTCCTTCTC
CTACCCCTCCCACGGCTGGATCTACCCCGGCCCCGTGGTGCACGGCTACT
GGTTCCGCGAGGGCGCCAACACCGACCAGGACGCCCCCGTGGCCACCAAC
AACCCCGCCCGCGCCGTGTGGGAGGAGACCCGCGACCGCTTCCACCTGCT
GGGCGACCCCCACACCAAGAACTGCACCCTGTCCATCCGCGACGCCCGCC
GCTCCGACGCCGGCCGCTACTTCTTCCGCATGGAGAAGGGCTCCATCAAG
TGGAACTACAAGCACCACCGCCTGTCCGTGAACGTGACCGCCCTGACCCA
CCGCCCCAACATCCTGATCCCCGGCACCCTGGAGTCCGGCTGCCCCCAGA
ACCTGACCTGCTCCGTGCCCTGGGCCTGCGAGCAGGGCACCCCCCCCATG
ATCTCCTGGATCGGCACCTCCGTGTCCCCCCTGGACCCCTCCACCACCCG
CTCCTCCGTGCTGACCCTGATCCCCCAGCCCCAGGACCACGGCACCTCCC
TGACCTGCCAGGTGACCTTCCCCGGCGCCTCCGTGACCACCAACAAGACC
GTGCACCTGAACGTGTCCTACCCCCCCCAGAACCTGACCATGACCGTGTT
CCAGGGCGACGGCACCGTGTCCACCGTGCTGGGCAACGGCTCCTCCCTGT
CCCTGCCCGAGGGCCAGTCCCTGCGCCTGGTGTGCGCCGTGGACGCCGTG
GACTCCAACCCCCCCGCCCGCCTGTCCCTGTCCTGGCGCGGCCTGACCCT
GTGCCCCTCCCAGCCCTCCAACCCCGGCGTGCTGGAGCTGCCCTGGGTGC
ACCTGCGCGACGCCGCCGAGTTCACCTGCCGCGCCCAGAACCCCCTGGGC
TCCCAGCAGGTGTACCTGAACGTGTCCCTGCAGTCCAAGGCCACCTCCGG
CGTGACCCAGGGCGTGGTGGGCGGCGCCGGCGCCACCGCCCTGGTGTTCC
TGTCCTTCTGCGTGATCTTCGTGGTGGTGCGCTCCTGCCGCAAGAAGTCC
GCCCGCCCCGCCGCCGGCGTGGGCGACACCGGCATCGAGGACGCCAACGC
CGTGCGCGGCTCCGCCTCCCAGGGCCCCCTGACCGAGCCCTGGGCCGAGG
ACTCCCCCCCCGACCAGCCCCCCCCCGCCTCCGCCCGCTCCTCCGTGGGC
GAGGGCGAGCTGCAGTACGCCTCCCTGTCCTTCCAGATGGTGAAGCCCTG
GGACTCCCGCGGCCAGGAGGCCACCGACACCGAGTACTCCGAGATCAAGA
TCCACCGCTAATCTAGAACGCGTTTAATTAA
The Siglec-9-Fc protein used for the protein
immunization had the following amino acid
sequence:
(SEQ ID NO: 11)
QTSKLLTMQSSVTVQEGLCVHVPCSFSYPSHGWIYPGPVVHGYWFREGAN
TDQDAPVATNNPARAVWEETRDRFHLLGDPHTKNCTLSIRDARRSDAGRY
FFRMEKGSIKWNYKHHRLSVNVTALTHRPNILIPGTLESGCPQNLTCSVP
WACEQGTPPMISWIGTSVSPLDPSTTRSSVLTLIPQPQDHGTSLTCQVTF
PGASVTTNKTVHLNVSYPPQNLTMTVFQGDGTVSTVLGNGSSLSLPEGQS
LRLVCAVDAVDSNPPARLSLSWRGLTLCPSQPSNPGVLELPWVHLRDAAE
FTCRAQNPLGSQQVYLNVSLQSKATSGVTQGVDKTHTCPPCPAPELLGGP
SVFLFPPKPKDTLMISRTPEVTCVWDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVNLTCLVKGFYPSDIAVEWESNGQPEN
NYKTTPPVLDSDGSFFLNSTLTVDKSRWQQGNVFSCSVMHEALHNHYTQK
SLSLS.

TABLE 1
Amino acid sequences for anti-Siglec-9
antibody 68D4.
68D4

| | | |
|---|---|---|
| SEQ ID | VHCDR1 | SYDMT |
| NO: 1 | (Kabat) | |
| SEQ ID | VHCDR2 | IIYTGGLTYYASWAKG |
| NO: 2 | (Kabat) | |
| SEQ ID | VHCDR3 | EVGDNNNF |
| NO: 3 | (Kabat) | |
| SEQ ID | VLCDR1 | QASESVDSGLA |
| NO: 4 | (Kabat) | |
| SEQ ID | VLCDR2 | DDTDLAS |
| NO: 5 | (Kabat) | |
| SEQ ID | VLCDR3 | QYTDYGRTYVGT |
| NO: 6 | (Kabat) | |
| SEQ ID | VH | QSLEESGGRLVKPDETLTITCTVSGIDLSSYDMT |
| NO: 7 | | WVRQAPGKGLEWIGIIYTGGLTYYASWAKGRFTI |
| | | SKTSTTVDLKITSPTTEDTATYFCAREVGDNNNF |
| | | WGPGTLVTVSS |
| SEQ ID | VL | DVVMTQTPASVSEPVGGTVTIKCQASESVDSGLA |
| NO: 8 | | WYHQKPGQPPKLLIYDDTDLASGVPSRFKGSRSG |
| | | TAHTLTISDLECADAATYYCQYTDYGRTYVGTFG |
| | | GGTEVVIK |
| SEQ ID | Full | QSLEESGGRLVKPDETLTITCTVSGIDLSSYDMT |
| NO: 28 | length | WVRQAPGKGLEWIGIIYTGGLTYYASWAKGRFTI |
| | heavy | SKTSTTVDLKITSPTTEDTATYFCAREVGDNNNF |
| | chain | WGPGTLVTVSSGQPKAPSVFPLAPCCGDTPSSTV |
| | | TLGCLVKGYLPEPVTVTWNSGTLTNGVRTFPSVR |
| | | QSSGLYSLSSWSVTSSSQPVTCNVAHPATNTKVD |
| | | KTVAPSTCSKPTCPPPELLGGPSVFIFPPKPKDT |
| | | LMISRTPEVTCVWDVSQDDPEVQFTWYINNEQVR |
| | | TARPPLREQQFNSTIRVVSTLPIAHQDWLRGKEF |
| | | KCKVHNKALPAPIEKTISKARGQPLEPKVYTMGP |
| | | PREELSSRSVSLTCMINGFYPSDISVEWEKNGKA |
| | | EDNYKTTPAVLDSDGSYFLYSKLSVPTSEWQRGD |
| | | VFTCSVMHEALHNHYTQKSISRSPGK |
| SEQ ID | Full | DVVMTQTPASVSEPVGGTVTIKCQASESVDSGLA |
| NO: 29 | length | WYHQKPGQPPKLLIYDDTDLASGVPSRFKGSRSG |
| | light | TAHTLTISDLECADAATYYCQYTDYGRTYVGTFG |
| | chain | GGTEVVIKGDPVAPTVLIFPPAADQVATGTVTIV |
| | | CVANKYFPDVTVTWEVDGTTQTTGIENSKTPQNS |
| | | ADCTYNLSSTLTLTSTQYNSHKEYTCKVTQGTTS |
| | | VVQSFNRGDC |

### Example 1b

NZW rabbits were immunized by DNA-immunization or by injection of Siglec-9-Fc-protein and antibody repertoire expression libraries were derived from lymph node and spleen B cells by cloning the immunoglobulin light chain and heavy chain variable region into an expression cassette providing the rabbit immunoglobulin constant heavy region and a transmembrane domain derived from human CD8. Screening of the antibody libraries was performed after transduction of the library in HEK 293T cells followed by antigen-specific sorting using fluorescently labeled Siglec-9. This sort yielded more than 1000 Siglec-9-specific-antibody-expressing cell clones which were further propagated for downstream analysis of antibody properties. Siglec-9-specific antibodies with high affinity and cross-reactivity to Siglec-7 were then sub-cloned into expression vectors for soluble antibody expression and expressed after transient transfection in HEK 293F cells. Antibodies were then characterized in the various assays after purification based on protein G.

The Siglec-9 DNA sequence used for genetic immunization and the Siglec-9-Fc protein used for the protein immunization were the same as described for Example 1a (SEQ ID NO: 10 and SEQ ID NO: 11).

TABLE 2
Amino acid sequences for anti-Sigiec-9
antibody 224B1.
224B1

| | | |
|---|---|---|
| SEQ ID | VHCDR1 | SYYMS |
| NO: 30 | (Kabat) | |
| SEQ ID | VHCDR2 | YIWTDGSAHYATWTKG |
| NO: 31 | (Kabat) | |
| SEQ ID | VHCDR3 | ANSGSHTYIEKFNL |
| NO: 32 | (Kabat) | |
| SEQ ID | VLCDR1 | QASPSINDELS |
| NO: 33 | (Kabat) | |
| SEQ ID | VLCDR2 | EASALES |
| NO: 34 | (Kabat) | |
| SEQ ID | VLCDR3 | QCTYGSRSDISYGNT |
| NO: 35 | (Kabat) | |
| SEQ ID | VH | QSVEESGGRLVTPETPLTLTCTASGFSLSSYYMSWV |
| NO: 36 | | RQAPGKGLEWIGYIWTDGSAHYATWTKGRFTISKTS |
| | | STTVDLKITSPTTEDTATYFCVRANSGSHTYIEKFN |
| | | LWGPGTLVTVSS |
| SEQ ID | VL | DVVMTQTPASVEATVGGSVTIKCQASPSINDELSWY |
| NO: 37 | | QQKPGQRPTLLIYEASALESGVPSRFKGSGSGTEFT |
| | | LTIGDLECADAATYYCQCTYGSRSDISYGNTFGGGT |
| | | EVVVKG |
| SEQ ID | Full | QSVEESGGRLVTPETPLTLTCTASGFSLSSYYMSWV |
| NO: 38 | length | RQAPGKGLEWIGYIWTDGSAHYATWTKGRFTISKTS |
| | heavy | STTVDLKITSPTTEDTATYFCVRANSGSHTYIEKFN |
| | chain | LWGPGTLVTVSSGQPKAPSVFPLAPCCGDTPSSTVT |
| | | LGCLVKGYLPEPVTVTWNSGTLTNGVRTFPSVRQSS |
| | | GLYSLSSVVSVTSSSQPVTCNVAHPATNTKVDKTVA |
| | | PSTCSKPTCPPPELLGGPSVFIFPPKPKDTLMISRT |
| | | PEVTCWVDVSQDDPEVQFTWYINNEQVRTARPPLRE |
| | | QQFNSTIRWSTLPIAHQDWLRGKEFKCKVHNKALPA |
| | | PIEKTISKARGQPLEPKVYTMGPPREELSSRSVSLT |
| | | CMINGFYPSDISVEWEKNGKAEDNYKTTPAVLDSDG |
| | | SYFLYSKLSVPTSEWQRGDVFTCSVMHEALHNHYTQ |
| | | KSISRSPGK |
| SEQ ID | Full | DVVMTQTPASVEATVGGSVTIKCQASPSINDELSWY |
| NO: 39 | length | QQKPGQRPTLLIYEASALESGVPSRFKGSGSGTEFT |
| | light | LTIGDLECADAATYYCQCTYGSRSDISYGNTFGGGT |
| | chain | EWVKGDPVAPTVLIFPPAADQVATGTVTIVCVANKY |
| | | FPDVTVTWEVDGTTQTTGIENSKTPQNSADCTYNLS |
| | | STLTLTSTQYNSHKEYTCKVTQGTTSVVQSFNRGD |
| | | C |

### Assays

### Comparative Antibodies

mAbA (WO 2017/153433), 5C6 (WO 2017/075432); mAbA was cloned as described in WO 2017/153433 as a mouse-human chimera. The mouse VH and VL domains were fused with human constant domains for IgG expression, resulting mAbA antibody was detected by a secondary anti-human IgG. 5C6 (WO 2017/075432) was cloned as a mouse-rabbit chimera. The mouse VH and VL domains were fused with rabbit constant domains for IgG expression. The resulting 5C6 antibody was detected by the same secondary anti-rabbit antibody as the here described rabbit antibodies. Negative control: 53C3. 53C3 was raised in a rabbit by immunisation with an unrelated peptide.

TABLE 3
Amino acid sequences for comparative anti-
Siglec-9 antibodies mAbA (WO 2017/153433)
and 5C6 (WO 2017/075432)
mAbA (WO 2017/153433)

| | | |
|---|---|---|
| SEQ ID | VHCDR1 | SYWMH |
| NO: 12 | (Kabat) | |
| SEQ ID | VHCDR2 | EINPSNGHTNYNEKFES |
| NO: 13 | (Kabat) | |
| SEQ ID | VHCDR3 | GVESYDFDDALDY |
| NO: 14 | (Kabat) | |
| SEQ ID | VLCDR1 | RASQDINNYLN |
| NO: 15 | (Kabat) | |
| SEQ ID | VLCDR2 | YTSRLHS |
| NO: 16 | (Kabat) | |
| SEQ ID | VLCDR3 | QQGNTLPFT |
| NO: 17 | (Kabat) | |
| SEQ ID | VH | QVQLQQPGAELVKPGSPVKLSCKASYFTFTSYWMHW |
| NO: 18 | | VRQRPGQGLEWIGEINPSNGHTNYNEKFESKATLTV |
| | | DRSSSTAYMQLSSLTSEDSAVFYCANGVESYDFDDA |
| | | LDYWGQGTSVTVSS |
| SEQ ID | VL | DIQMTQTTSSLSASLGDRVTISCRASQDINNYLNWY |
| NO: 19 | | QQKPDGTIKLLIYYTSRLHSGVPSRFSGSGSGTDYS |
| | | LTINNLEQEDIATYFCQQGNTLPFTFGGGTKLEIK |
| SEQ ID | Full | QVQLQQPGAELVKPGSPVKLSCKASYFTFTSYWMHW |
| NO: 40 | length | VRQRPGQGLEWIGEINPSNGHTNYNEKFESKATLTV |
| | heavy | DRSSSTAYMQLSSLTSEDSAVFYCANGVESYDFDDA |
| | chain | LDYWGQGTSVTVSSASTKGPSVFPLAPSSKSTSGGT |
| | | AALGCLVKDYFPEPVTVSWNSGALTSGVHTFPAVLQ |
| | | SSGLYSLSSWTVPSSSLGTQTYICNVNHKPSNTKVD |
| | | KKVEPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEYK |
| | | CKVSNKALPAPIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| SEQ ID | Full | DIQMTQTTSSLSASLGDRVTISCRASQDINNYLNWY |
| NO: 41 | length | QQKPDGTIKLLIYYTSRLHSGVPSRFSGSGSGTDYS |
| | light | LTINNLEQEDIATYFCQQGNTLPFTFGGGTKLEIKR |
| | chain | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |

5C6 (WO 2017/075432)

| | | |
|---|---|---|
| SEQ ID | VHCDR1 | TDYNIH |
| NO: 20 | (Kabat) | |
| SEQ ID | VHCDR2 | YIYPYNGDTGYNQKFQN |
| NO: 21 | (Kabat) | |
| SEQ ID | VHCDR3 | GNYLYYYAMDY |
| NO: 22 | (Kabat) | |
| SEQ ID | VLCDR1 | SSSQSLVHSNGNTYLH |
| NO: 23 | (Kabat) | |
| SEQ ID | VLCDR2 | KVSNRFS |
| NO: 24 | (Kabat) | |
| SEQ ID | VLCDR3 | SQNTHVPLT |
| NO: 25 | (Kabat) | |
| SEQ ID | VH | EVQLQQSGPELVKPGASVRISCKASGYTFTDYNIHW |
| NO: 26 | | VKQSQGKSLEWIGYIYPYNGDTGYNQKFQNKATLTV |
| | | DNSSSTAYMELRSLTSEDSAVYYCANGNYLYYYAMD |
| | | YWGQGTSVTVSS |
| SEQ ID | VL | DVVMTQTPLSLPVSLGDQVSISCSSSQSLVHSNGNT |
| NO: 27 | | YLHWYLQKSGQSPKLLIYKVSNRFSGVPDRFRGSGS |
| | | GTDFTLKISRVEAEDLGVYFCSQNTHVPLTFGAGTK |
| | | LELK |
| SEQ ID | Full | EVQLQQSGPELVKPGASVRISCKASGYTFTDYNIHW |
| NO: 42 | length | VKQSQGKSLEWIGYIYPYNGDTGYNQKFQNKATLTV |
| | heavy | DNSSSTAYMELRSLTSEDSAVYYCANGNYLYYYAMD |
| | chain | YWGQGTSVTVSSGQPKAPSVFPLAPCCGDTPSSTVT |
| | | LGCLVKGYLPEPVTVTWNSGTLTNGVRTFPSVRQSS |
| | | GLYSLSSVVSVTSSSQPVTCNVAHPATNTKVDKTVA |
| | | PSTCSKPTCPPPELLGGPSVFIFPPKPKDTLMISRT |
| | | PEVTCVWDVSQDDPEVQFTWYINNEQVRTARPPLRE |
| | | QQFNSTIRVVSTLPIAHQDWLRGKEFKCKVHNKALP |
| | | APIEKTISKARGQPLEPKVYTMGPPREELSSRSVSL |
| | | TCMINGFYPSDISVEWEKNGKAEDNYKTTPAVLDSD |
| | | GSYFLYSKLSVPTSEWQRGDVFTCSVMHEALHNHYT |
| | | QKSISRSPGK |
| SEQ ID | Full | DVVMTQTPLSLPVSLGDQVSISCSSSQSLVHSNGNT |
| NO: 43 | length | YLHWYLQKSGQSPKLLIYKVSNRFSGVPDRFRGSGS |
| | light | GTDFTLKISRVEAEDLGVYFCSQNTHVPLTFGAGTK |
| | chain | LELKGDPVAPTVLIFPPAADQVATGTVTIVCVANKY |
| | | FPDVTVTWEVDGTTQTTGIENSKTPQNSADCTYNLS |
| | | STLTLTSTQYNSHKEYTCKVTQGTTSWQSFNRGDC |

### Example 2

### Specificity/Cross-Reactivity of Siglec-9-Specific Antibody to Siglec-5, -7, -8, -9 and -15

### Material and Methods

Specificity towards Siglec-5, Siglec-7, Siglec-8, Siglec-15 and Siglec-9 was assayed in enzyme-linked immunosorbent assay (ELISA) using 96 well half-area ELISA plates (Corning). Siglec-5-Fc (R&D systems), Siglec-7-Fc (in-house production, Siglec-7-Fc protein used had the following amino acid sequence:

(SEQ ID NO: 59))
QKSNRKDYSLTMQSSVTVQEGMCVHVRCSFSYPVDSQTDSDPVHGYWFRA
GNDISWKAPVATNNPAWAVQEETRDRFHLLGDPQTKNCTLSIRDARMSDA
GRYFFRMEKGNIKWNYKYDQLSVNVTALTHRPNILIPGTLESGCFQNLTC
SVPWACEQGTPPMISWMGTSVSPLHPSTTRSSVLTLIPQPQHHGTSLTCQ
VTLPGAGVTTNRTIQLNVSYPPQNLTVTVFQGEGTASTALGNSSSLSVLE
GQSLRLVCAVDSNPPARLSWTWRSLTLYPSQPSNPLVLELQVHLGDEGEF
TCRAQNSLGSQHVSLNLSLQQEYTGKMRPVSGVLLVDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVYTLPPSREEMTKNQVNLTCLVKGFYPSDIAVEWESN
GQPENNYKTTPPVLDSDGSFFLNSTLTVDKSRWQQGNVFSCSVMHEALHN
HYTQKSLSLS, Siglec-8-Fc (R&D systems), Siglec-15-
Fc (R&D systems) and Siglec-9-Fc (in-house
production, SEQ ID NO: 11) were coated at 1 μg/ml
in 50 mM carbonate-bicarbonate buffer pH 9.6 for
15-20 hours at 4° C.

Antigens were washed away (Tecan Hydrospeed) and plates were blocked with 5% skimmed milk in phosphate-buffered saline (PBS). After washing the plates, antibodies were added at 5 μg/ml (33 nM) in 0.5% milk PBS in triplicates. Plates with antibodies were incubated for 1 hour at room temperature. Plates were washed and bound antibodies were detected by horseradish peroxidase (HRP)-labeled polyclonal anti-rabbit IgG antibody (Biolegend) or anti-human Fab fragment for mAbA (Sigma Aldrich). 3,3′,5,5′-Tetramethylbenzidine (TMB; Sigma Aldrich) development was stopped by addition of sulfuric acid and absorbance was detected at 450 nm (Tecan, CM INFINITE MONO 200).

### Results

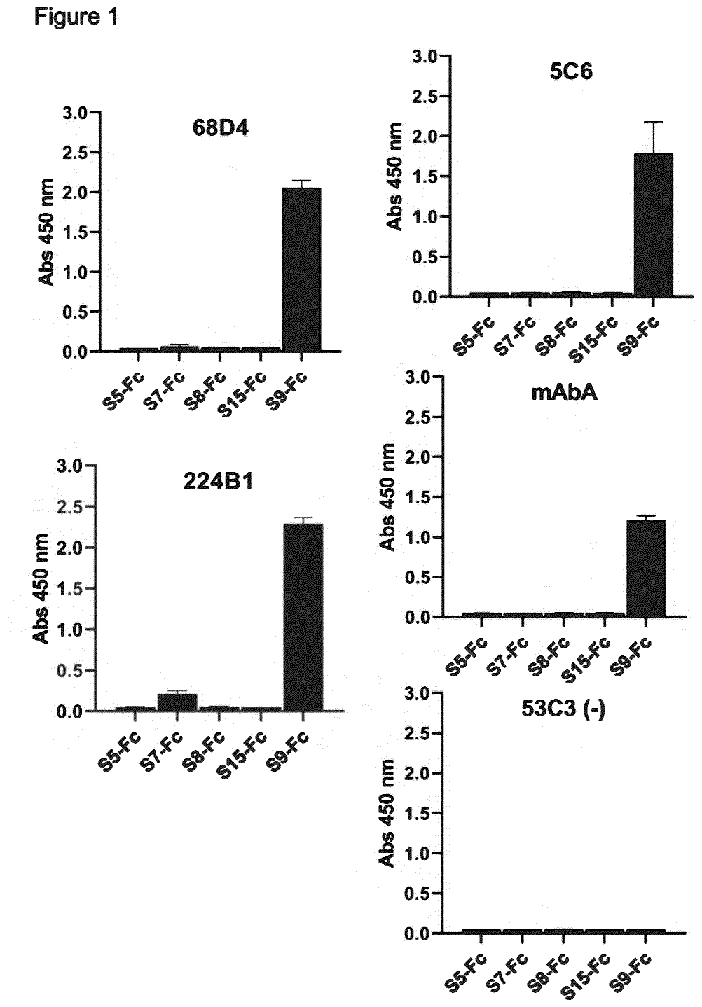

The monoclonal antibody 68D4 shows specific binding towards Siglec-9, but no cross-reactivity towards the closely related Siglec-5, Siglec-7, Siglec-8 and Siglec-15. The two comparative antibodies mAbA and 5C6 show the same behaviour. The negative control 53C3 displays no reactivity towards any of the tested antigens. The monoclonal antibody 224B1 shows specific binding towards Siglec-9 and Siglec-7, but no cross-reactivity towards the closely related Siglec-5, Siglec-8 and Siglec-15 (FIG. 1).

### Example 3

### EC50 Measurements of Siglec-9-Specific Antibody to Siglec-9 Protein in Enzyme-Linked Immunosorbent Assay (ELISA)

### Material and Methods

Antibody binding affinity towards Siglec-9 was measured by a dilution series of the analysed antibodies on a fixed concentration of Siglec-9 protein in ELISA. To this end, Siglec-9 was coated at 1 μg/ml in 50 mM carbonate-bicarbonate buffer pH 9.6 for 15-20 hours at 4° C. Antigen solution was washed away (Tecan Hydrospeed) and plates were blocked with 5% skimmed milk in phosphate-buffered saline (PBS) for 1 hour at room temperature. Plates were washed and antibodies were added at concentrations of 0-420 nM. After incubation at room temperature for 1 hour plates were washed once more. Bound antibodies were detected by addition of horseradish peroxidase (HRP)-coupled secondary antibodies. Rabbit antibodies were detected by polyclonal anti-rabbit IgG antibody (Biolegend), mAbA by anti-human Fab fragment (Sigma Aldrich). After a last washing step, plates were developed by the addition of 3,3′,5,5′-Tetramethylbenzidine (TMB; Sigma Aldrich). Reaction was stopped by addition of sulfuric acid. Absorbance was detected at 450 nm (Tecan, CM INFINITE MONO 200). Data were fitted and EC50 values were determined by three-parameter analysis in GraphPad Prism (GraphPad Software)

### Results

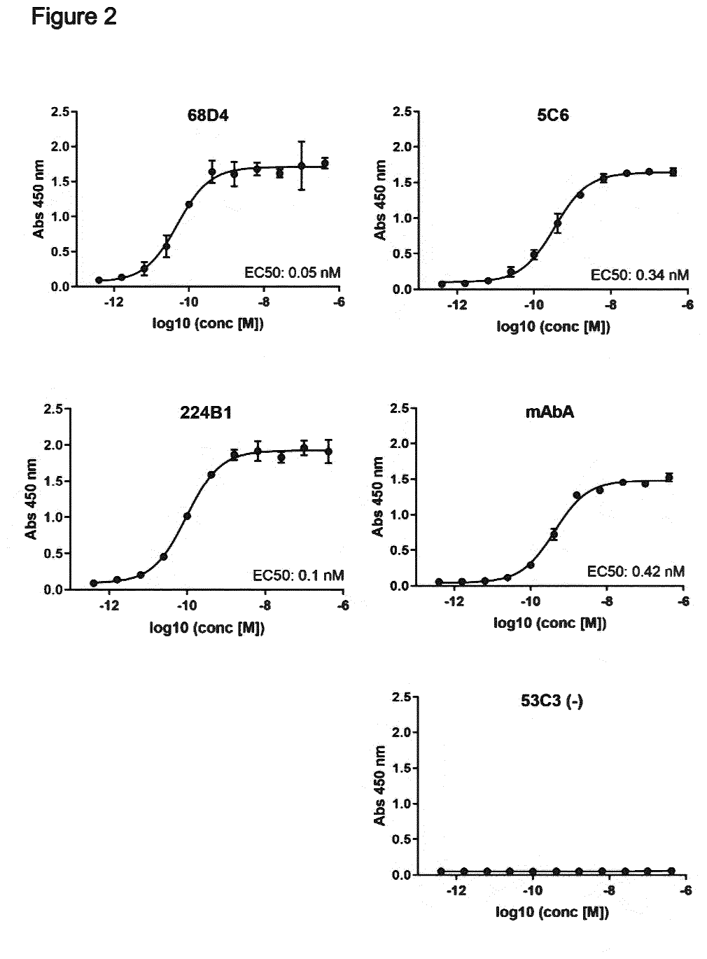

The monoclonal antibodies 68D4 and 224B1 show a much stronger avidity to Siglec-9 as compared to mAbA and 5C6 (FIG. 2).

### Example 4

### Affinity Measurements by Surface Plasmon Resonance (SPR)

### Materials and Methods

K_{D} determination was performed by SPR by individually coating the antibodies to HC2000M chips via sulpho-NHS/EDC coupling chemistry. Chips were afterwards blocked with ethanolamine. Antigen was applied in one of the three different concentrations (20 nM, 100 nM and 500 nM) with a contact time of 240 s, followed by a dissociation phase of 120 s. After each antigen incubation, the bound antigen was removed from the antibodies with 10 mM glycine at pH 2.0. All kinetic parameters (kₒₙ, k_{off} and K_{D}) were determined using a kinetic fitting program (LSA Kinetics, Carterra).

### Results

The monoclonal antibody 68D4 shows strong avidity with a faster on-rate and slower off-rate as the comparative antibody mAbA, resulting in an ˜16.7 fold better dissociation constant. The monoclonal antibody 224B1 shows strong avidity.

TABLE 4
Kinetic parameters as determined by SPR

| Antibody | K_{D} (M) | kₒₙ (M⁻¹ s⁻¹) | k_{off} (s⁻¹) |
|---|---|---|---|
| 68D4 | 1.50E−10 | 6.40E+05 | 9.60E−05 |
| 224B1 | 5.10E−09 | 2.30E+05 | 1.20E−03 |
| mAbA | 2.50E−09 | 2.10E+05 | 5.30E−04 |

### Example 5

### Binding of Siglec-9-Specific Antibody to Human Monocytes

### Material and Methods

Binding of antibodies to human monocytes was evaluated due to the fact that monocytes express the target, Siglec-9 strongly and in a uniform manner. Therefore, human monocytes are an excellent and relevant ex vivo model to characterize binding of Siglec-9-specific antibodies.

Human ex vivo PBMC were stained with fluorescence-labeled (allophycocyanin, APC) antibody to the monocyte marker CD14 (Biolegend) in assay buffer (0.5% (v/v) heat-inactivated fetal bovine serum (Gibco), 2 mM EDTA in phosphate-buffered saline). Subsequently, cells were incubated with antibodies under evaluation at various concentrations (67 nM-0.38 pM). After a washing step cells were incubated with fluorescence-labeled (Brilliant Violet 421™) secondary antibody against human IgG (mAbA) or rabbit IgG (all other monoclonal antibodies under evaluation; both secondary antibodies from Biolegend).

Binding of Siglec-9-specific antibodies to monocytes (CD14-positive life cells) was analysed with a CytoFLEX flow cytometer (Beckham Coulter's). The mean fluorescence intensity of the secondary antibody staining in the CD14-positive population at each dilution was determined using Flow Jo software (BD). Datapoints generated by this analysis were fitted and EC50 values were determined by three-parameter analysis in GraphPad Prism (GraphPad Software)

### Results

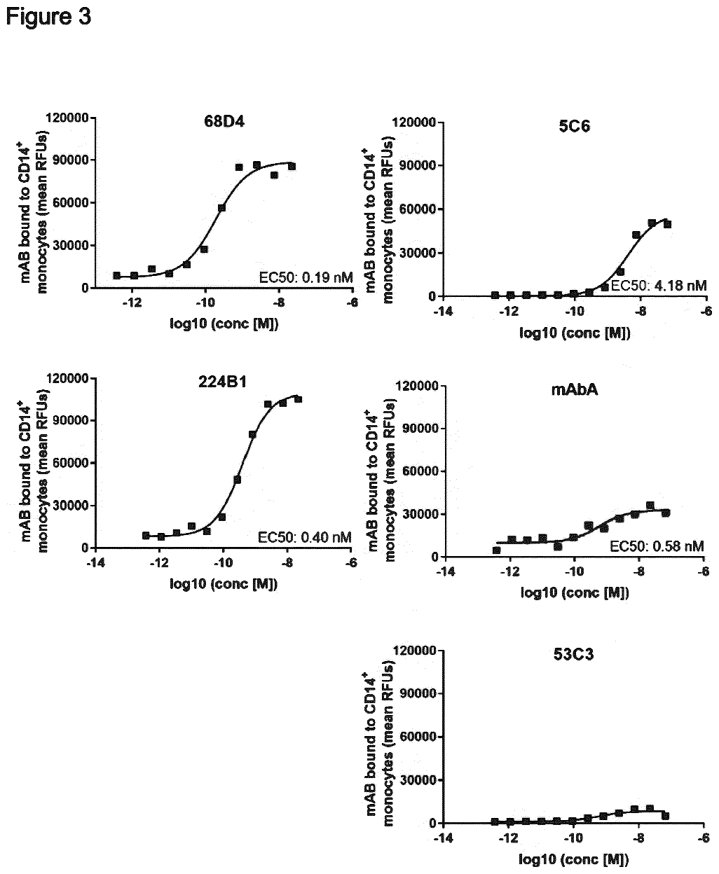

In the relevant cellular context of human CD14+ monocytes, monoclonal antibody 68D4 shows an approximately 3-fold stronger avidity to monocyte-expressed Siglec-9 as compared to mAbA. Compared to monoclonal antibody 5C6, 68D4 appears to bind approximatively 22-fold stronger. In the relevant cellular context of human CD14+ monocytes, monoclonal antibody 224B1 shows a similar avidity to monocyte-expressed Siglec-9 as compared to mAbA. Compared to monoclonal antibody 5C6, 224B1 appears to bind approximatively 10-fold stronger (FIG. 3).

### Example 6

### Blocking of Ligand Binding to Siglec-9

### Materials and Methods

To analyse if anti-Siglec-9 antibodies recognize the ligand-binding domain and block the binding of Siglec-9 to its ligands, sialic acids, we performed a cell-based assay where the binding of sialic-acid expressing A549 tumour cells to Siglec-9-Fc is evaluated in ELISA. ELISA-plates were coated with 3 μg/ml Protein-A (Thermo Fisher Scientific) overnight at 4° C. Plates were blocked with PBS, 1% BSA for 1 h, and incubated with 5 μg/ml Siglec-9-Fc for 1 h at RT. Anti-Siglec-9 antibodies at different concentrations (starting at 8 μg/ml or 53 nM); were mixed 1:1 with Calcein-AM (Thermo Fisher Scientific) labeled A549 tumour cells. The mixture was added to the ELISA plate and incubated for 1 h at 4° C. After washing of the plate, cells that were bound to the plate were lysed with PBS, 1% Triton-X 100 and released fluorescence was measured at an excitation of 485 nm and emission of 535 nm with a Synergy H1 (BioTek). Data were fitted by three-parameter analysis in GraphPad Prism (GraphPad Software).

### Results

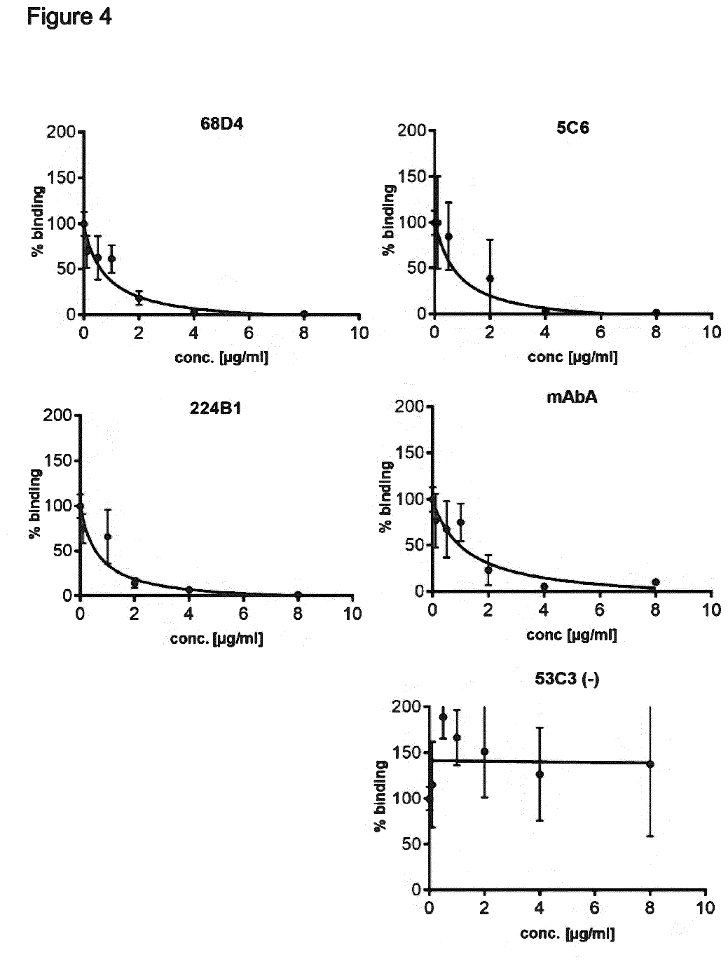

Monoclonal antibodies 68D4 and 224B1 block the binding of sialic-acid expressing A549 tumour cells to Siglec-9 in a concentration dependent manner and in a comparable extend to comparative monoclonal antibodies mAbA and 5C6 (FIG. 4).

### Example 7

### T Cell Activation Assay

### Materials and Methods

For the T cell activation assay, cells from Siglec-9 transgenic mice (Stanczak et al., J Clin Invest. 2018 Nov. 1; 128(11):4912-4923) were used. In these mice, Siglec-9 expression is induced with Cre recombinase by excision of a GFP-stop cassette in front of the Siglec-9 gene. Cre expression is driven by the CD4 promoter. Expression of Siglec-9 in these T cells results in a phenotype where the stimulation via triggering of the T cell receptor and a co-stimulatory signal via CD28 receptor alone is not sufficient to activate the cell, a third signal that disrupts Siglec-9-mediated inhibition is needed.

For the T cell activation assay, 96-well flat bottom plates were coated with 0.5 μg/ml anti-CD3 and 1 μg/ml anti-CD28 over night at 4° C. or for 2 h at 37° C. Splenocytes and lymph node cells were isolated from Siglec-9 transgenic mice. Erythrocytes were lysed and cells were labeled with Cell Trace Violet (Thermo Fisher Scientific). 250.000 cells/well were added with or without antibodies under evaluation at various concentrations (0.25 μg/ml-0.0125 μg/ml; 1.7 nM-83 pM). Cells were cultured for 48 h and afterwards stained for T cell markers CD3, CD4 and CD8 and for activation markers CD69 and CD25. Proliferation of cells was assessed by analysing the dilution of Cell Trace Violet. Cells were analysed with a CytoFLEX flow cytometer (Beckham Coulter's). Expansion Index was calculated with Flow Jo software (BD). Activation of cells was measured by mean fluorescence intensity of CD69 and CD25 on T cells. Data was depicted using GraphPad Prism (GraphPad Software).

### Results

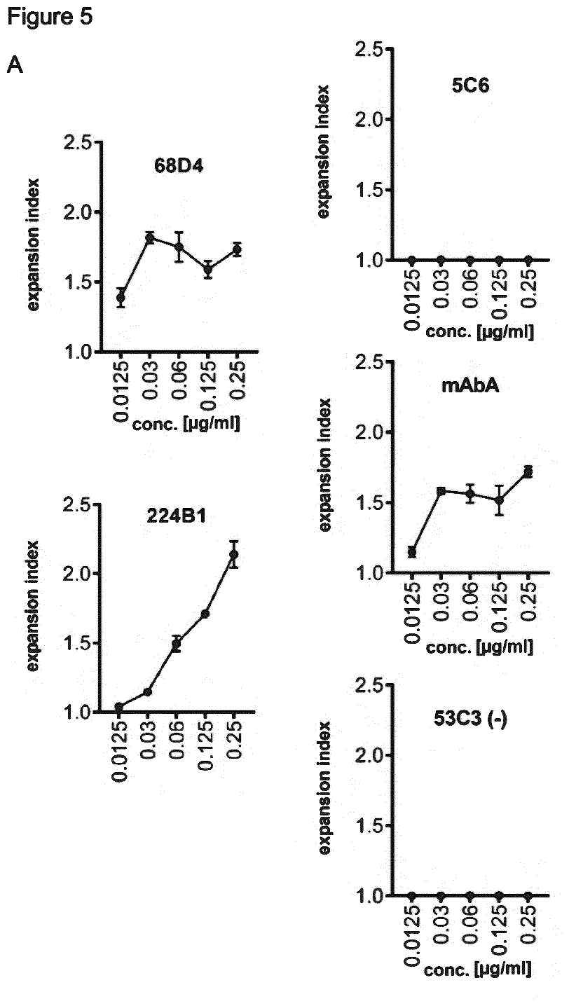

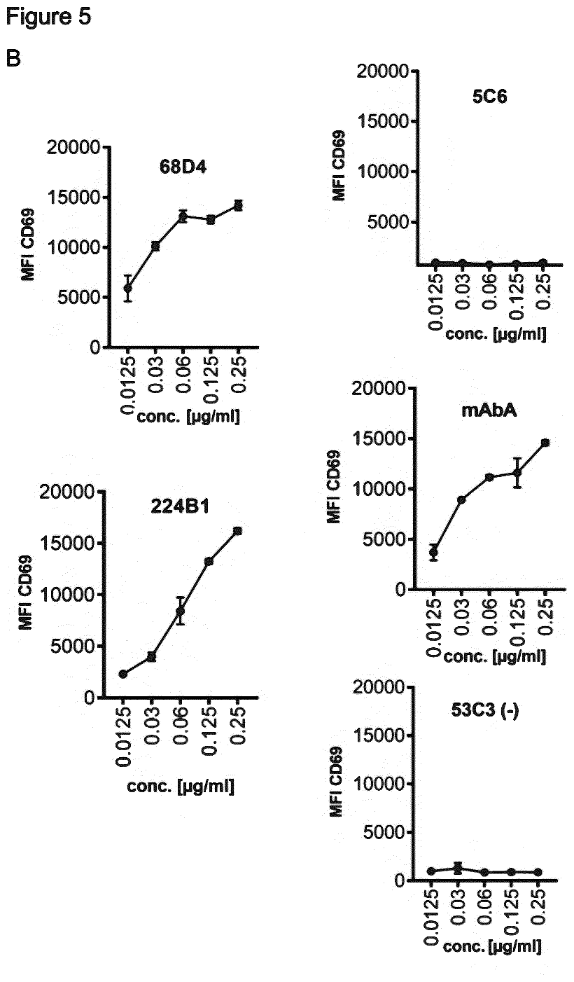

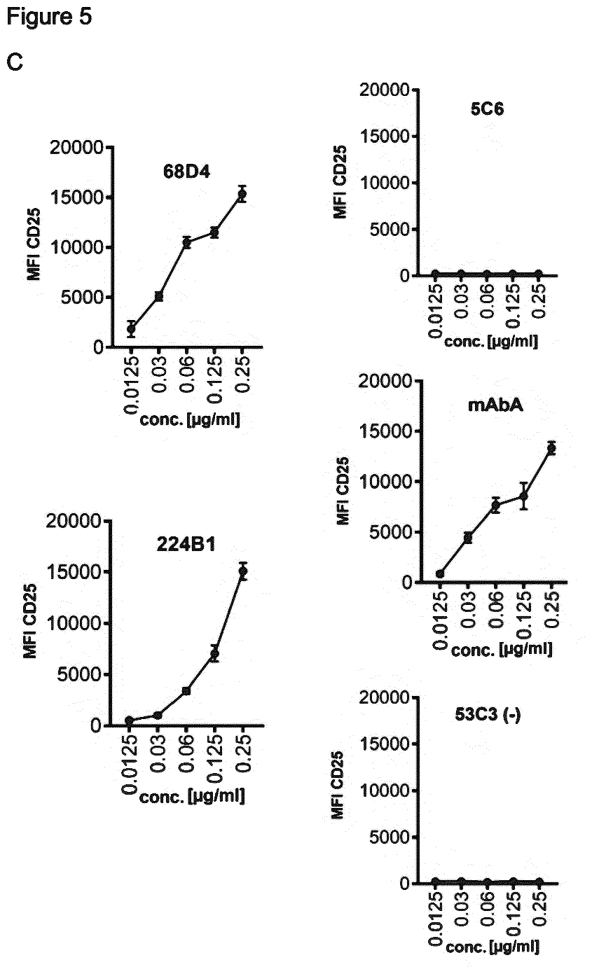

CD8+ T cells co-stimulated with monoclonal antibody 68D4 or 224B1 responded in a concentration dependent manner by proliferation and up-regulation of activation markers CD69 and CD25. Monoclonal antibody 68D4 showed a stronger proliferation and activation at lower concentrations as compared to comparative antibody mAbA. Monoclonal antibody 224B1 showed a stronger proliferation at higher concentrations as compared to comparative antibody mAbA. Activation factors seem to be comparable for both antibodies 224B1 and mAbA. Monoclonal antibody 5C6 appeared to be neither stimulating nor activating (FIG. 5).

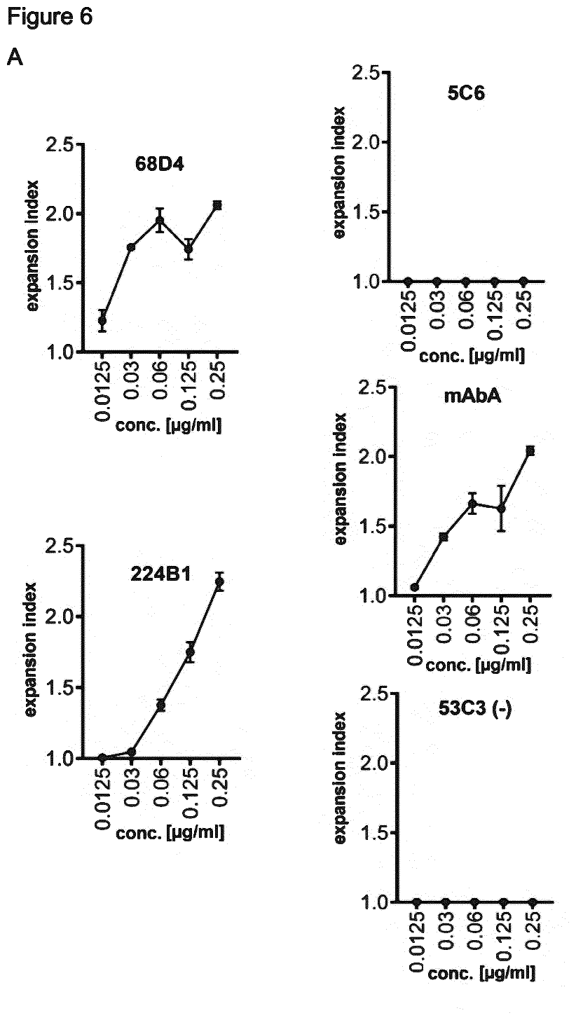

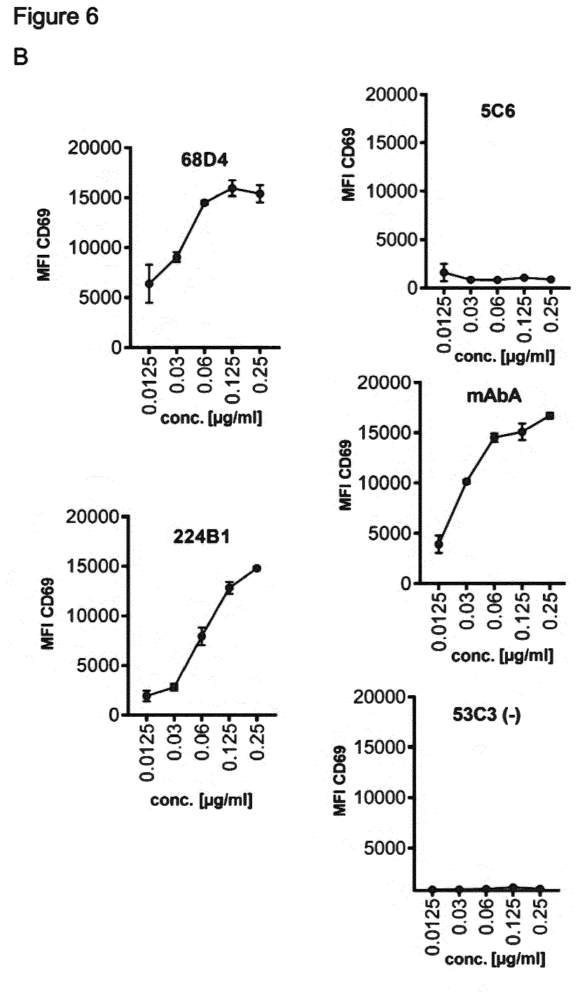

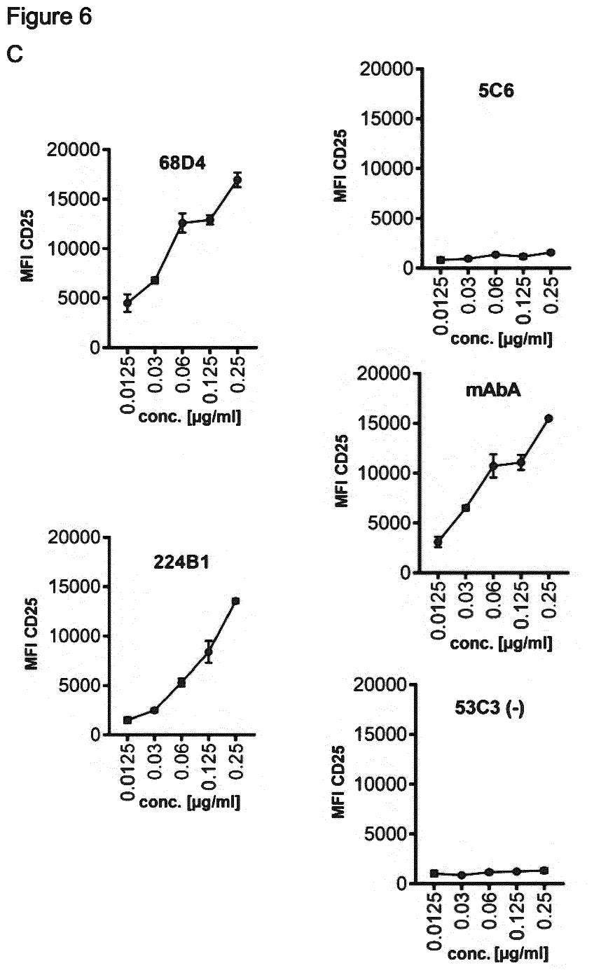

Likewise, in CD4+ T cells monoclonal antibody 68D4 showed a much stronger proliferation at lower concentrations as compared to mAbA and monoclonal antibody 224B1 showed a much stronger proliferation at higher concentrations as compared to mAbA. As in CD8+ T cells monoclonal antibody 5C6 failed to elicit a stimulatory or activatory response. In all cases, the stronger proliferation is also accompanied by a stronger upregulation of the activation markers CD69 and CD25 (FIG. 6).

### Example 8

### Activation of Natural Killer Cell Activity

### Materials and Methods

NK cell killing assay was performed using a human NK cell line that overexpresses Siglec-9.

NK92 cells were stably transfected with a plasmid containing Siglec-9 and GFP (NK92-Siglec-9). Control cells were stably transfected with GFP only (NK92-GFP). NK92 cells were activated over night with 20 ng/ml IL-15 and co-cultured with Calcein-AM labeled K562 cells in a 3:1 ratio for 4 h with or without an antibody dilution series in 96-well V-bottom plates. Cells were pelleted by centrifugation and supernatant was transferred to black 96-well flat bottom plates. Lysis of K562 cells was measured as the released Calcein-AM in the supernatant with an excitation at 485 nm and emission at 535 nm with a Synergy H1 (BioTek). Specific lysis was calculated as: (lysis test well−spontaneous lysis)/(maximal lysis−spontaneous lysis)×100%. For spontaneous cell lysis, K562 cells only were use. For maximal lysis, Triton X-100 was added to K562 cells. Data was analysed in GraphPad Prism (GraphPad Software).

### Results

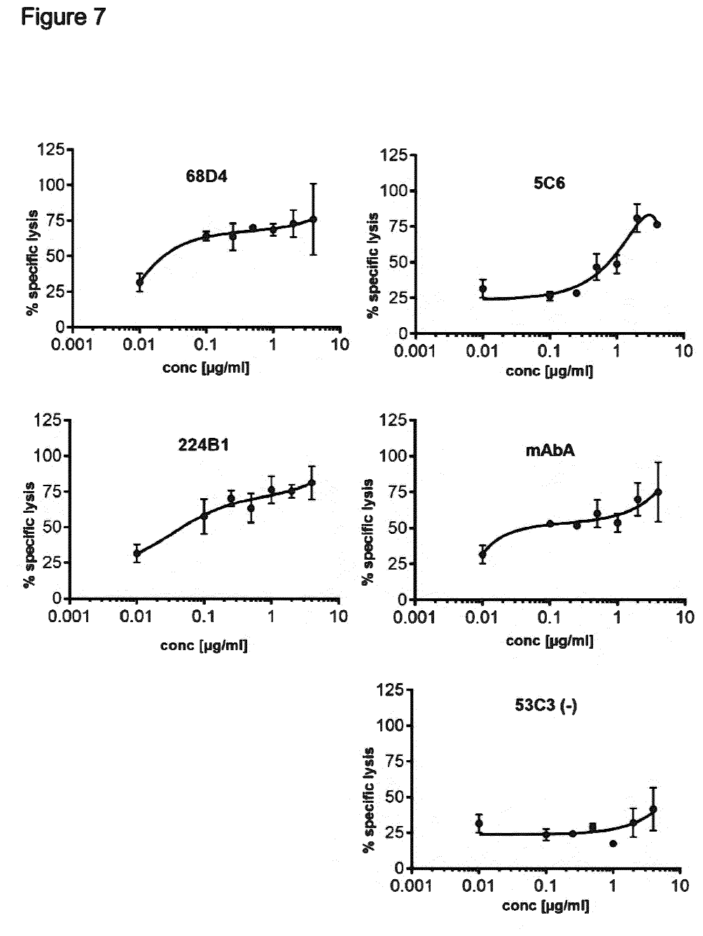

Monoclonal antibody 68D4 showed a concentration dependent activation of NK cell-mediated killing activity. The specific cell lysis reached higher values already at lower concentrations, when compared to comparative monoclonal antibodies mAbA and 5C6. The latter antibody gave rise to cell lysis only at the highest concentrations tested. Monoclonal antibody 224B1 showed a concentration dependent activation of NK cell-mediated killing activity. The specific cell lysis reached comparable values as comparative monoclonal antibody mAbA (FIG. 7).

### Example 9

### Humanization of Rabbit Antibody 68D4

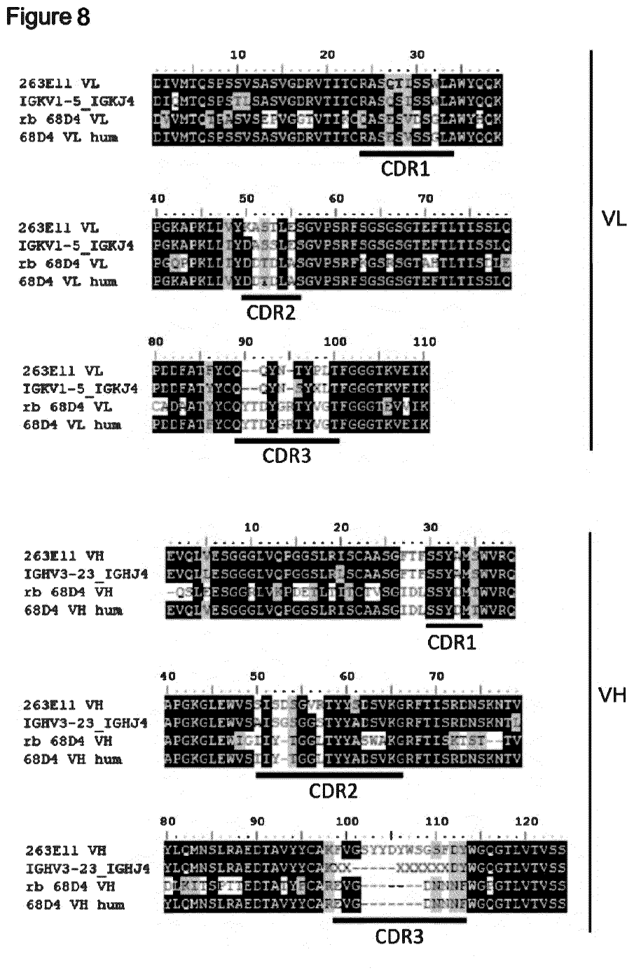

Human IgG 263E11 was picked from Memo Therapeutics AG human IgG database due to its close similarity to 68D4. As a first measure CDRs of rabbit immunoglobulin 68D4 were grafted onto the 263E11 variable domain frameworks. Further adjustments were made to framework positions: in the heavy chain Kabat positions 27, 28 and 29 as well as position 94 were replaced with rabbit amino acids (compare FIG. 8). Additionally, some rabbit CDR positions were changed to the human amino acids. In the light chain variable domain Kabat positions 24 and 30 in CDR1 were replaced by the human amino acid side chains (FIG. 8). In the heavy chain CDR2 Kabat positions 61, 62 and 63 were replaced by the human amino acids. Genes for the resulting heavy and light chain sequences were synthesised and cloned into IgG1/kappa expression vectors. Humanized 68D4 (68D4-hum) was expressed and purified from H EK293F cells. 68D4-hum was compared to its parent rb 68D4 and to mAbA in ELISA essentially following the protocol described in example 3.

As antigen extra-cellular domain of Siglec-9 fused to a C-terminal his-tag was used:

(SEQ ID NO: 44)
QTSKLLTMQSSVTVQEGLCVHVPCSFSYPSHGWIYPGPVVHGYWFREGAN
TDQDAPVATNNPARAVWEETRDRFHLLGDPHTKNCTLSIRDARRSDAGRY
FFRMEKGSIKWNYKHHRLSVNVTALTHRPNILIPGTLESGCPQNLTCSVP
WACEQGTPPMISWIGTSVSPLDPSTTRSSVLTLIPQPQDHGTSLTCQVTF
PGASVTTNKTVHLNVSYPPQNLTMTVFQGDGTVSTVLGNGSSLSLPEGQS
LRLVCAVDAVDSNPPARLSLSWRGLTLCPSQPSNPGVLELPWVHLRDAAE
FTCRAQNPLGSQQVYLNVSLQSKATSGVTQGVDHHHHHH. For
detection of bound IgG either polyclonal anti-
rabbit IgG antibody (68D4; Biolegend, #406401),
or anti-human Fey fragment (humanized 68D4 and
mAbA; Jackson Immuno Research laboratories,
#109-035-098) were used.

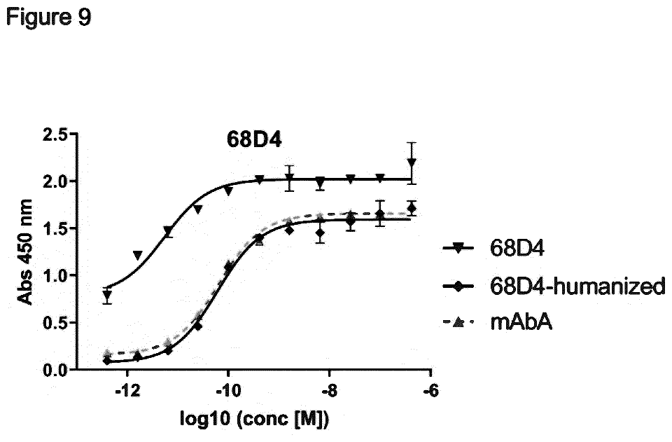

As a result it was observed that humanization of 68D4 led to a high affinity Siglec-9 binder with slightly reduced binding capacity as compared to rabbit 68D4 (FIG. 9). Siglec-9 binding capacity is comparable to mAbA, a mouse variable domain expressed with a human IgG Fc.

TABLE 5
Amino acid sequences for humanized anti-Siglec-9 antibody 68D4.
68D4-hum

| | | |
|---|---|---|
| SEQ ID | VHCDR1 | SYDMT |
| NO: 1 | (Kabat) | |
| SEQ ID | VHCDR2 | IIYTGGLTYYADSVKG |
| NO: 45 | (Kabat) | |
| SEQ ID | VHCDR3 | EVGDNNNF |
| NO: 3 | (Kabat) | |
| SEQ ID | VLCDR1 | RASESVSSGLA |
| NO: 46 | (Kabat) | |
| SEQ ID | VLCDR2 | DDTDLAS |
| NO: 5 | (Kabat) | |
| SEQ ID | VLCDR3 | QYTDYGRTYVGT |
| NO: 6 | (Kabat) | |
| SEQ ID | VH | EVQLVESGGGLVQPGGSLRISCAASGIDLSSYDMTW |
| NO: 47 | | VRQAPGKGLEWVSIIYTGGLTYYADSVKGRFTISRDN |
| | | SKNTVYLQMNSLRAEDTAVYYCAREVGDNNNFWGQ |
| | | GTLVTVSS |
| SEQ ID | VL | DIVMTQSPSSVSASVGDRVTITCRASESVSSGLAWYQ |
| NO: 48 | | QKPGKAPKLLVYDDTDLASGVPSRFSGSGSGTEFTLT |
| | | ISSLQPDDFATFYCQYTDYGRTYVGTFGGGTKVEIK |
| SEQ ID | Full | EVQLVESGGGLVQPGGSLRISCAASGIDLSSYDMTW |
| NO: 49 | length | VRQAPGKGLEWVSIIYTGGLTYYADSVKGRFTISRDN |
| | heavy | SKNTVYLQMNSLRAEDTAVYYCAREVGDNNNFWGQ |
| | chain | GTLVTVSSASTKGPSVFPLAPSSKSTSGGTAALGCLV |
| | | KDYFPEPVTVSWNSGALTSGVHTFPAVLQSSGLYSL |
| | | SSVVTVPSSSLGTQTYICNVNHKPSNTKVDKKVEPKS |
| | | CDKTHTCPPCPAPELLGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSREEMTKNQVSL |
| | | TCLVKGFYPSDIAVEWESNGQPENNYKTTPPVLDSD |
| | | GSFFLYSKLTVDKSRWQQGNVFSCSVMHEALHNHYT |
| | | QKSLSLSPGK |
| SEQ ID | Full | DIVMTQSPSSVSASVGDRVTITCRASESVSSGLAWYQ |
| NO: 50 | length | QKPGKAPKLLVYDDTDLASGVPSRFSGSGSGTEFTLT |
| | light | ISSLQPDDFATFYCQYTDYGRTYVGTFGGGTKVEIKR |
| | chain | TVAAPSVFIFPPSDEQLKSGTASVVCLLNNFYPREAK |
| | | VQWKVDNALQSGNSQESVTEQDSKDSTYSLSSTLTL |
| | | SKADYEKHKVYACEVTHQGLSSPVTKSFNRGEC |

TABLE 6
CDR amino acid sequences for rabbit/
humanized anti-Siglec-9 antibody 68D4.
68D4 (rabbit/humanized)

| | | |
|---|---|---|
| SEQ ID | VHCDR1 | SYDMT |
| NO: 1 | (Kabat) | |
| SEQ ID | VHCDR2 | IIYTGGLTYYAX₁X₂X₃KG, wherein X₁ is |
| NO: 51 | (Kabat) | D or S; X₂ is S or W; and X₃ is V or A |
| SEQ ID | VHCDR3 | EVGDNNNF |
| NO: 3 | (Kabat) | |
| SEQ ID | VLCDR1 | X₄ASESVX₅SGLA, wherein X₄ is R or Q; |
| NO: 52 | (Kabat) | and X₅ is S or D |
| SEQ ID | VLCDR2 | DDTDLAS |
| NO: 5 | (Kabat) | |
| SEQ ID | VLCDR3 | QYTDYGRTYVGT |
| NO: 6 | (Kabat) | |

1. An anti-Siglec-9 antibody molecule or an anti-Siglec-9 binding fragment thereof comprising:
a heavy chain variable region (VH) comprising a heavy chain complementarity determining region 1 (VHCDR1) amino acid sequence of SEQ ID NO: 1 or a sequence having one, two, three, or four amino acid substitutions, a heavy chain complementarity determining region 2 (VHCDR2) amino acid sequence of SEQ ID NO: 51 or a sequence having one, two, three, or four amino acid substitutions, and a heavy chain complementarity determining region 3 (VHCDR3) amino acid sequence of SEQ ID NO: 3 or a sequence having one, two, three, or four amino acid substitutions; and
a light chain variable region (VL) comprising a light chain complementarity determining region 1 (VLCDR1) amino acid sequence of SEQ ID NO: 52 or a sequence having one, two, three, or four amino acid substitutions, a light chain complementarity determining region 2 (VLCDR2) amino acid sequence of SEQ ID NO: 5 or a sequence having one, two, three, or four amino acid substitutions, and a light chain complementarity determining region 3 (VLCDR3) amino acid sequence of SEQ ID NO: 6 or a sequence having one, two, three, or four amino acid substitutions.

2. The antibody molecule or binding fragment thereof of claim 1, comprising:
(i) a heavy chain variable region (VH) comprising a heavy chain complementarity determining region 1 (VHCDR1) amino acid sequence of SEQ ID NO: 1 or a sequence having one, two, three, or four amino acid substitutions, a heavy chain complementarity determining region 2 (VHCDR2) amino acid sequence of SEQ ID NO: 2 or a sequence having one, two, three, or four amino acid substitutions, and a heavy chain complementarity determining region 3 (VHCDR3) amino acid sequence of SEQ ID NO: 3 or a sequence having one, two, three, or four amino acid substitutions; and
a light chain variable region (VL) comprising a light chain complementarity determining region 1 (VLCDR1) amino acid sequence of SEQ ID NO: 4 or a sequence having one, two, three, or four amino acid substitutions, a light chain complementarity determining region 2 (VLCDR2) amino acid sequence of SEQ ID NO: 5 or a sequence having one, two, three, or four amino acid substitutions, and a light chain complementarity determining region 3 (VLCDR3) amino acid sequence of SEQ ID NO: 6 or a sequence having one, two, three, or four amino acid substitutions;
or
(ii) a heavy chain variable region (VH) comprising a heavy chain complementarity determining region 1 (VHCDR1) amino acid sequence of SEQ ID NO: 1 or a sequence having one, two, three, or four amino acid substitutions, a heavy chain complementarity determining region 2 (VHCDR2) amino acid sequence of SEQ ID NO: 45 or a sequence having one, two, three, or four amino acid substitutions, and a heavy chain complementarity determining region 3 (VHCDR3) amino acid sequence of SEQ ID NO: 3 or a sequence having one, two, three, or four amino acid substitutions; and
a light chain variable region (VL) comprising a light chain complementarity determining region 1 (VLCDR1) amino acid sequence of SEQ ID NO: 46 or a sequence having one, two, three, or four amino acid substitutions, a light chain complementarity determining region 2 (VLCDR2) amino acid sequence of SEQ ID NO: 5 or a sequence having one, two, three, or four amino acid substitutions, and a light chain complementarity determining region 3 (VLCDR3) amino acid sequence of SEQ ID NO: 6 or a sequence having one, two, three, or four amino acid substitutions.

3. The antibody molecule or binding fragment thereof of claim 1, comprising:
a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 51, and a heavy chain complementarity determining region 3 (VHCDR3) amino acid sequence of SEQ ID NO: 3; and
a light chain variable region (VL) comprising a light chain complementarity determining region 1 (VLCDR1) amino acid sequence of SEQ ID NO: 52, a light chain complementarity determining region 2 (VLCDR2) amino acid sequence of SEQ ID NO: 5, and a light chain complementarity determining region 3 (VLCDR3) amino acid sequence of SEQ ID NO: 6.

4. The antibody molecule or binding fragment thereof of claim 1, comprising:
(i) a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 2, and a heavy chain complementarity determining region 3 (VHCDR3) amino acid sequence of SEQ ID NO: 3; and
a light chain variable region (VL) comprising a light chain complementarity determining region 1 (VLCDR1) amino acid sequence of SEQ ID NO: 4, a light chain complementarity determining region 2 (VLCDR2) amino acid sequence of SEQ ID NO: 5, and a light chain complementarity determining region 3 (VLCDR3) amino acid sequence of SEQ ID NO: 6;
or
(ii) a heavy chain variable region (VH) comprising a heavy chain complementarity determining region 1 (VHCDR1) amino acid sequence of SEQ ID NO: 1, a heavy chain complementarity determining region 2 (VHCDR2) amino acid sequence of SEQ ID NO: 45, and a heavy chain complementarity determining region 3 (VHCDR3) amino acid sequence of SEQ ID NO: 3; and
a light chain variable region (VL) comprising a light chain complementarity determining region 1 (VLCDR1) amino acid sequence of SEQ ID NO: 46, a light chain complementarity determining region 2 (VLCDR2) amino acid sequence of SEQ ID NO: 5, and a light chain complementarity determining region 3 (VLCDR3) amino acid sequence of SEQ ID NO: 6.

5. The antibody molecule or binding fragment thereof of any one of claims 1-4, comprising:
(i) a heavy chain variable region (VH) comprising the amino acid sequence of SEQ ID NO: 7, and a light chain variable region (VL) comprising the amino acid sequence of SEQ ID NO: 8;
or
(ii) a heavy chain variable region (VH) comprising the amino acid sequence of SEQ ID NO: 47, and a light chain variable region (VL) comprising the amino acid sequence of SEQ ID NO: 48.

6. The antibody molecule or binding fragment thereof of any one of claims 1-5, comprising one or more (e.g., 2, 3, 4, 5, 6, 7, 8 or 9) of the following properties:
(i) binds to human Siglec-9 with a EC50 of less than about 10 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM, e.g., when the antibody molecule or binding fragment thereof is tested as a bivalent molecule using ELISA, e.g., as described in Example 3;
(ii) binds to human Siglec-9 with a dissociation constant (KD) of less than about 10 nM, 1 nM, 0.9 nM, 0.8 nM, 0.7 nM, 0.6 nM, 0.5 nM, 0.4 nM, 0.3 nM, 0.2 nM or 0.1 nM, when the antibody molecule or binding fragment thereof is tested as a bivalent molecule using surface plasmon resonance, e.g. Carterra LSA, e.g., as described in Example 4;
(iii) binds to human CD14+ monocytes with a EC50 of less than about 10 nM, 1 nM, 0.9 nM, 0.8 nM, 0.7 nM, 0.6 nM, 0.5 nM, 0.4 nM, 0.3 nM, 0.2 nM or 0.1 nM, e.g., when the antibody molecule or binding fragment thereof is tested as a bivalent molecule using flow cytometer, e.g., as described in Example 5;
(vi) inhibits interaction between Siglec-9 and one or more Siglec-9 ligands, e.g., inhibits the binding of sialic-acid expressing A549 tumour cells to Siglec-9, e.g., when the antibody molecule or binding fragment thereof is tested as a bivalent molecule using ELISA, e.g., as described in Example 6;
(v) increases proliferation of anti-CD3/anti-CD28 stimulated CD8+ T cells, e.g., when the antibody molecule or binding fragment thereof is tested as a bivalent molecule in a T cell activation assay using Cell Trace Violet and flow cytometer, e.g., as described in Example 7;
(vi) upregulates activation markers CD69 and CD25 of anti-CD3/anti-CD28 stimulated CD8+ T cells, e.g., when the antibody molecule or binding fragment thereof is tested as a bivalent molecule in a T cell activation assay using staining and flow cytometer, e.g., as described in Example 7;
(vii) increases proliferation of anti-CD3/anti-CD28 stimulated CD4+ T cells, e.g., when the antibody molecule or binding fragment thereof is tested as a bivalent molecule in a T cell activation assay using Cell Trace Violet and flow cytometer, e.g., as described in Example 7;
(viii) upregulates activation markers CD69 and CD25 of anti-CD3/anti-CD28 stimulated CD4+ T cells, e.g., when the antibody molecule or binding fragment thereof is tested as a bivalent molecule in a T cell activation assay using staining and flow cytometer, e.g., as described in Example 7; or
(ix) increases NK cell-mediated killing activity, e.g., increases killing of K562 cells by NK92 cells overexpressing Siglec-9, e.g., when the antibody molecule or binding fragment thereof is tested as a bivalent molecule using Calcein-AM labeled K562 cells, e.g., as described in Example 8.

7. An antibody molecule or a binding fragment thereof that competes for binding to human Siglec-9 with the antibody molecule or binding fragment thereof of any one of claims 1-6.

8. The antibody molecule or binding fragment thereof of claim 7 that competes for binding to the same epitope of human Siglec-9 with the antibody molecule or binding fragment thereof of any one of claims 1-6.

9. A pharmaceutical composition comprising the antibody molecule or binding fragment thereof of any one of claims 1-8 and a pharmaceutically acceptable carrier, excipient or stabilizer.

10. An antibody molecule or binding fragment thereof according to any one of claims 1-8 or a pharmaceutical composition according to claim 9 for use in the treatment of a disease selected from the group consisting of cancer, acute and chronic hepatitis B.

11. An antibody molecule or binding fragment thereof according to any one of claims 1-8 or a pharmaceutical composition according to claim 9 for the use of claim 10, wherein the cancer is selected from the group consisting of non small cell lung cancer, colorectal cancer, breast cancer, epithelial ovarian cancer, hepatocellular carcinoma and prostate cancer.

12. A nucleic acid encoding the antibody heavy and/or light chain variable region of the antibody molecule or binding fragment thereof of any one of claims 1-8.

13. An expression vector comprising the nucleic acid of claim 12.

14. A host cell comprising the nucleic acid of claim 12 or the expression vector of claim 13.

15. A method of producing an antibody molecule, the method comprising culturing the host cell of claim 14 under conditions suitable for gene expression.
